(19) United States
(12) Patent Application Publication
(10) Pub. No.: US 2017/0320939 A1
(43) Pub. Date: Nov. 9, 2017

(54) COLL2-1 PEPTIDE AND ITS NITRATED FORM AS THERAPEUTIC TARGETS FOR OSTEOARTHRITIS TREATMENT

(71) Applicants: Université de Liège, Angleur (BE); Université Paris Descartes, Paris (FR); Assistance Publique Hopitaux de Paris, Paris (FR); Institut National de la Santé et de la Recherche Médicale, Paris (FR)

(72) Inventors: Yves HENROTIN, Boncelles (BE); Cécile LAMBERT, Louveigne (BE); Didier BORDERIE, Paris (FR); François RANNOU, Colombes (FR)

(73) Assignees: Université de Liège, Angleur (BE); Université Paris Descartes, Paris (FR); Assistance Publique Hopitaux de Paris, Paris (FR); Institut National de la Santé et de la Recherche Médicale, Paris (FR)

(21) Appl. No.: 15/624,801

(22) Filed: Jun. 16, 2017

Related U.S. Application Data
(63) Continuation-in-part of application No. PCT/EP2015/078867, filed on Dec. 7, 2015.

(30) Foreign Application Priority Data
Dec. 19, 2014 (EP) 14199098.9

(51) Int. Cl. C07K 16/18; A61K 39/00

(52) U.S. Cl. CPC C07K 16/18; C07K 2317/76; C07K 2317/622; C07K 2317/55; A61K 2039/505; C07K 2317/34; C07K 2317/24; A61K 2039/507; C07K 2317/52

(57) ABSTRACT

The present invention provides a medicament comprising as active ingredient an inhibitor of Coll2-1 peptide activity and/or an inhibitor of Coll2-1NO₂ peptide activity for use in the prevention and/or treatment of osteoarthritis. Further, the present invention provides a medicament comprising as active ingredient an inhibitor of Coll2-1 peptide activity and/or an inhibitor of Coll2-1NO₂ peptide activity for use in the prevention and/or treatment of rheumatic and musculoskeletal diseases (RMDs).

### FIELD OF THE INVENTION

The present invention refers to a medicament for use in the prevention and/or treatment of osteoarthritis

### BACKGROUND OF THE INVENTION

Osteoarthritis (OA) is the most common joint disease and it is a major cause of joint pain and disability in the aging population. Its etiology is multifactorial (i.e., age, obesity, joint injury, genetic predisposition), and the pathophysiologic process affects the entirety of the joint. Destruction of articular cartilage, sclerosis of subchondral bone, and formation of osteophytes, synovial inflammation, and ligament and meniscal damage constitute the main features characterizing OA.

Synovial inflammation plays a critical role in the symptoms and structural progression of osteoarthritis. Several studies have revealed that crosstalk between the joint tissues, communicated at the cellular levels within an innate immunity inflammatory network, can promote synovitis and cartilage degradation. Endogenous molecular products derived from cellular stress and extracellular matrix disruption can function as DAMPs (Damage-associated molecular pattern) to induce inflammatory response and pro-catabolic events in vitro and produce synovitis and cartilage degradation in vivo via PRRs (Pattern recognition receptors).

Under the action of pro-inflammatory cytokines, synoviocytes and chondrocytes can produce reactive oxygen species and NO. These one can induce post-translational modifications (for example, formation of carbonyl groups or nitration of matrix proteins including collagen type II).

Synovial neovascularization is another important feature of the inflamed synovial membrane, resulting from an imbalance between pro- and anti-angiogenic factors. The neovascularization may be largely driven by synovitis.

Cartilage matrix is synthesized, organized, maintained and degraded by a sparse population of chondrocytes. The properties of cartilage are critically dependent upon the structure and integrity of the extracellular matrix (ECM). In a normal cartilage the anabolic and catabolic processes of ECM formation and degradation are well balanced. In joint diseases, such as rheumatoid arthritis (RA) and osteoarthritis (OA), the rate of degradation of the ECM often exceeds the rate of synthesis. Thereby the structural integrity and mechanical strength of the tissues is impaired, resulting in irreversible destruction of the joint structures.

Collagen degradation is one of the main features of cartilage breakdown during OA. Type II collagen is the principal component of extracellular matrix of articular cartilage comprising 15-25% of the wet weight and 90-95% of the total collagen content of the cartilage. Each molecule is composed mainly of a triple helix of three identical alpha chains and form fibrils stabilized by intermolecular crosslink. Damage to this fibrillar meshwork is a critical event in the pathogenesis of OA.

The type II collagen degradation product is a specific OA biomarker that can assess both disease progression and activity. Specific immunoassays have been developed to measure a specific peptide located in the triple helix, Coll2-1 and its nitrated form Coll2-1NO₂, a marker associated with local oxidative stress. These biomarkers have been studied in vivo in mouse, guinea pig and horse and also in healthy human and OA patients.

Degradation of collagen type II involves collagenases (MMP1, MMP8 and MP13). A characteristic collagenase cleavage site is found in the triple helical region of collagen type II between residues 775 and 776, which generates two fragments containing ¾ and ¼ of the intact collagen molecule. Antibodies, which recognize the C-terminal part of the COL2-¾ fragment and the N-terminal part of the COL2-¼ fragments, have been developed. It has been demonstrated that the COL2-¾ epitope but not the COL2-¼ epitopes have been found in circulation, probably due to a higher resistance to proteolysis of the COL2-¾ fragment. Specific immunoassays for detection of the COL2-¾ neoepitope in body fluids have been developed (U.S. Pat. No. 6,132,976). It has been reported that RA and OA patients assessed in a cross sectional study have elevated levels of this collagen type II derived marker.

The COL2-¾ and COL2-¼ fragments are approximately 75 kDa and 25 kDa respectively. U.S. Pat. No. 6,132,976 describes detection of collagen type II fragments in synovial fluid and serum utilizing an epitope located within the COL2-¾ fragment.

Fragments generated from the telopeptidic region (U.S. Pat. No. 5,641,837, U.S. Pat. No. 5,919,634, U.S. Pat. No. 6,342,361) also filtrate more readily into body fluids, however these fragments are not generated as a result of collagenase activity, which is believed to be responsible for the initial collagen breakdown seen in joint diseases.

Detection of other cartilage derived metabolites, such as free urinary pyridinoline, cartilage oligomeric matrix protein (COMP), hyaluronates, aggrecan and collagen type III fragments, arising from destruction of joint tissues affected by an inflammatory disease have also been reported (PCT application WO 01/38872).

The document WO 2003/076947 discloses a method for detecting and/or monitoring cartilage degradation. The method enables such detection by measuring in a biological sample a collagen type II fragment wherein all or a relevant part of the amino acid sequence HRGYPGLDG is contained, The method utilizes an immunoassay to detect fragments of collagen type II resulting from collagenase activity comprising an antibody directed against an epitope comprised in the amino acid sequence HRGYPGLDG (Coll2-1 peptide), located in the helical region of collagen type II. Thus the document WO 2003/076947 provides a method of qualitative or quantitative assay or collagen type II or fragments thereof in a biological sample comprising contacting said fragments with an immunological binding partner which is immunoreactive with an epitope comprised in the amino acid sequence HRGYPGLDG and detecting resulting immunoreaction.

The present inventors sought to provide means to alleviate, reduce or prevent the effects of Coll2-1 peptide and derived metabolites and to treat and/or prevent osteoarthritis.

### BRIEF SUMMARY OF THE INVENTION

When carrying out the studies according to the present invention the inventors investigated the effects of Coll2-1 peptide on the expression of inflammatory mediators (IL-8 and IL-6) and angiogenics factors (TSP1 and VEGF; TSP1: Thrombospondin-1; anti-angiogenic factor; VEGF: Vascular Endothelial Growth Factor; pro-angiogenic factor) as well as on the production of reactive oxygen species in OA synovial cells. In the presence of increasing doses of Coll2-1 peptide, they observed a increase of H₂O₂ production, a decrease of NO production and also a decrease of GSH in synovial cells. The expression of inflammatory mediators was also observed to be increased. Finally, in terms of angiogenesis, the present inventors observed an increase of VEGF, pro-angiogenic factor and a decrease of TSP1, anti-angiogenic factor, sign of an imbalance between pro- and anti-angiogenic factors.

The studies carried out according to the present invention highlights the pro-infammatory and immunomodulatory properties of Coll2-1 peptide. These results are critical. They demonstrate that the Coll2-1, a marker of cartilage degradation is also directly involved in the physiopathology of osteoarthritis and rheumatic arthritis. Therefore, a neutralization of Coll2-1 and Coll2-1NO₂, respectively, or a decrease of its release from cartilage could modulate local and systemic inflammatory and immune responses. In this context, Coll2-1 and Coll2-1NO₂, respectively, was found to represent a therapeutic target for a biotherapy.

None of the prior art documents discloses or suggests that the Coll2-1 peptide has an activity of enhancing type II collagen breakdown.

For treating and/or preventing osteoarthritis means which mediate the decrease of the Coll2-1 release or which neutralize the activity of Coll2-1 peptide are required but so far not provided in the prior art.

Therefore, the present invention provides a medicament comprising as active ingredient an inhibitor of Coll2-1 peptide activity and/or an inhibitor of Coll2-1NO₂ peptide activity for use in the prevention and/or treatment of osteoarthritis.

Further, the present invention provides a medicament comprising as active ingredient is an inhibitor of Coll2-1 peptide activity and/or an inhibitor of Coll2-1NO₂ peptide activity for use in the prevention and/or treatment of rheumatic and musculoskeletal diseases (RMDs)

The Coll2-1 peptide activity and/or an inhibitor of Coll2-1NO₂ peptide activity is due to Coll2-1 peptide and Coll2-1 NO₂ peptide, respectively, or a peptide comprising Coll2-1 peptide and Coll2-1 NO₂ peptide, respectively. The Coll2-1 peptide is consisting of the amino acid sequence HRGYPGLDG (SEQ ID NO: 1). The Coll2-1-NO₂ peptide is modified by NO₂-group. Peptides comprising Coll2-1 peptide are peptides comprising the amino acid sequence HRGYPGLDG. Peptides comprising Coll2-1NO₂ peptide are peptides comprising the amino acid sequence HRGYPGLDG-NO₂.

In a preferred embodiment of the medicament of the present invention the inhibitor of Coll2-1 or Coll2-1 NO₂ peptide activity inhibits or reduces the enhancing effects of Coll2-1 or Coll2-1 NO₂ peptide activity on any one or more or all of the following: the reactive oxygen species production (such as H₂O₂), the expression of angiogenic factors, the expression of proinflammatory cytokines (such as IL-6 and 1L-8) each in osteoarthritic synovial fibroblast cells.

In respect to the angiogenic factors the inhibitor of Coll2-1 or Coll2-1 NO₂ peptide activity inhibits or reduces the enhancing effects of Coll2-1 or Coll2-1 NO₂ peptide activity on expression of pro-angiogenic factors (VEGF); whereas the inhibitor of Coll2-1 or Coll2-1 NO₂ peptide activity inhibits, reduces or decreases the reducing effects of Coll2-1 or Coll2-1 NO₂ peptide activity on expression of anti-angiogenic factors (TSP1).

In another preferred embodiment of the medicament said inhibitor of Coll2-1 or Coll2-1NO₂ peptide activity is an immunological binding partner which is immune-reactive with an epitope comprised in the amino acid sequence HRGYPGLDG (SEQ ID NO: 1).

In a further preferred embodiment the immunological binding partner is selected from the group consisting of polyclonal antibodies, monoclonal antibodies, humanized antibodies, Fc fragments, Fab fragments, single chain antibodies (scFv), chimeric antibodies, biobetters, other antigen-specific antibody fragments specifically binding to Coll2-1 or Co112-1NO₂ peptide or other ligand receptor fragments specifically binding to coll2-1 or coll2-1NO₂ peptide.

In a particularly preferred embodiment the immunological binding partner is monoclonal or polyclonal antibody specifically binding to Coll2-1 or Coll2-1NO₂ peptide.

In another particularly preferred embodiment the medicament comprises a mixture of an immunological binding partner specifically binding to Coll2-1 peptide and an immunological binding partner specifically binding to Coll2-1NO₂ peptide.

The present invention also provides a method of prevention and/or treatment of osteoarthritis comprising the administration of a subject in need thereof an effective dose of an inhibitor of Coll2-1 peptide activity and/or an inhibitor of Coll2-1 NO₂ peptide activity.

Further, the present invention provides a method of prevention and/or treatment of rheumatic and musculoskeletal diseases (RMDs) comprising the administration of a subject in need thereof an effective dose of an inhibitor of Coll2-1 peptide activity and/or an inhibitor of Coll2-1NO₂ peptide activity.

In a preferred method of the present invention the active ingredient is an immunological binding partner which is immune-reactive with an epitope comprised in the amino acid sequence HRGYPGLDG (SEQ ID NO: 1). Further, preferred embodiments of the method of prevention and/or treatment of osteoarthritis are already described above for the respective medicament.

### BRIEF DESCRIPTION OF THE DRAWINGS

FIG. 1 consists of FIGS. 1A and 1B which show the effects of Coll2-1 (A) and Coll2-1NO₂ (B) peptides on H₂O₂ production in OA synovial fibroblast cells. Synovial fibroblast cells were cultured without (Ctl) or with increasing concentrations of Coll2-1 (0.45 and 4.5 nmol) or Coll2-1NO₂ (4 and 40 pmol) peptides during 24 hours. Results are expressed as mean±SEM (N=10). *P<0.05 and **P<0.01.

FIG. 2 consists of FIGS. 2A and 2B which show the effects of Coll2-1 (A) and Coll2-1NO₂ (B) peptides on GSH production in OA synovial fibroblast cells. Synovial fibroblast cells were cultured without (Ctl) or with increasing concentrations of Coll2-1 (0.45 and 4.5 nmol) or Coll2-1NO₂ (4 and 40 pmol) peptides during 24 hours. Results are expressed as mean±SEM (N=10). **P<0.01 and ***P<0.001.

FIG. 3 consists of FIGS. 3A and 3B which show the effects of Coll2-1 (A) and Coll2-1NO₂ (B) peptides on NO production in OA synovial fibroblast cells. Synovial fibroblast cells were cultured without (Ctl) or with increasing concentrations of Coll2-1 (0.45 and 4.5 nmol) or Coll2-1NO₂ (4 and 40 pmol) peptides during 24 hours. Results are expressed as mean±SEM (N=10). **P<0.01 and ***P<0.001.

FIG. 4 consists of FIGS. 4A and 4B which show the effects of Coll2-1 (A) and Coll2-1NO₂ (B) peptides on TSP1 expression in OA synovial fibroblast cells. Synovial fibroblast cells were cultured without (Ctl) or with increasing concentrations of Coll2-1 (0.45 and 4.5 nmol) or Coll2-1NO₂ (4 and 40 pmol) peptides during 24 hours. Total RNA was isolated in cellular extract and TSP1 mRNA expression was analyzed by Real Time PCR Technology. Results are expressed as mean±SEM (N=10 for Coll2-1 peptide and N=5 for Coll21NO₂ peptide). **P<0.01.

FIG. 5 consists of FIGS. 5A and 5B which show the effects of peptides Coll2-1(A) and Coll2-1NO₂(B) on VEGF expression in OA synovial fibroblast cells. Synovial fibroblast cells were cultured without (Ctl) or with increasing concentrations of Coll2-1 (0.45 and 4.5 nmol) or Coll2-1NO₂ (4 and 40 pmol) peptides during 24 hours. Total RNA was isolated in cellular extract and VEGF mRNA expression was analyzed by Real Time FOR technology. Results are expressed as mean±SEM (N=10 for Coll2-1 peptide and N=5 for Coll2-1NO₂ peptide).

FIG. 6 consists of FIGS. 6A, 6B and 6C which show the effects of peptides Coll2-1 and Coll2-1NO₂ on inflammatory mediators expression in OA synovial fibroblast cells, Synovial fibroblast cells were cultured without (Ctl) or with increasing concentrations of Coll2-1 (0.45 and 4.5 nmol) or Coll2-1NO₂ (4 and 40 pmol) peptides during 24 hours. Total RNA was isolated in cellular extract and IL-8 (A-B) and IL-6 (C) mRNA expression was analyzed by Real Time PCR technology. Results are expressed as mean±SEM (N=5 except for effects of Coll2-1 peptide on 1L-8 expression, N=10), *P<0.05 and **P<0.01.

FIG. 7 consists of FIGS. 7A, 7B and 7C which show the competitive inhibition of AS0619 with Coll2-1 peptide and the effect on oxidative stress parameters H₂O₂ (A), GSH (B) and NO (C). Synovial fibroblast cells were cultured without (Ctl) or with Coll2-1 (4.5 nmol) peptide, in the presence or absence of AS0619 during 24 hours. Total RNA was isolated in cellular extract and IL-8 mRNA expression was analyzed by Real Time PCR technology. Results are expressed as mean±SEM (N=1 patient, in triplicate). *P<0.05.

FIG. 8 shows the competitive inhibition of AS0619 with Coll2-1 peptide and the effect on IL8 expression. Synovial fibroblast cells were cultured without (Ctl) or with Coll2-1 (4.5 nmol) peptide, in the presence or absence of AS0619 during 24 hours. Results are expressed as mean±SEM (N=1 patient, in triplicate). *P<0.05; **P<0.01 and ***P<0.01.

FIG. 9 shows the absorbance of polyclonal antibody directed to ¹⁰⁸HRGYPGLDG¹¹⁶ peptide after purification on Protein A columns at wavelengths from 220 nm to 600 nm.

FIG. 10 consists of FIGS. 10A, 10B, and 10C which show the competitive inhibition of D37 with Coll2-1NO₂ peptide and the effect on oxidative stress parameters H₂O₂ (A), GSH (B) and NO (C). Synovial fibroblast cells were cultured without (Ctl) or with Coll2-1NO₂ (40 pM) peptide, in the presence or absence of D37 during 24 hours. Results are expressed as mean±SEM (N=3). *P<0.05 and **P<0.01.

FIG. 11 shows the competitive inhibition of D37 with Coll2-1NO₂ peptide and the effect on VEGF expression. Synovial fibroblast cells were cultured without (Ctl) or with Coll2-1NO₂ (40 pM) peptide, in the presence or absence of D37 during 24 hours. Results are expressed as mean±SEM (N=3).

FIG. 12 shows the effect of Coll2-1, in the presence or absence of oxidative stress inhibitors (I), on the phosphorylation of p65 and IκB-α.

FIG. 13 shows the effect of CLI-095 on IL-8 expression. Synoviocytes were pre-treated 1 hour with CLI-095 (500 nM, 1 and 2.5 μM) before a 24 hours treatment with Coll2-1 at 4.5 nmol. Results are expressed as mean±SEM (N=4).

FIG. 14 shows the Final design for the in-vivo study

FIG. 15 shows the body weight evolution

FIG. 16 shows Arthritis visual score after S.C. injection. Peptide injection was compared to CIA.

FIG. 17 consists of FIGS. 17A and 17B which show Knee diameter measured after S.C. injection: A) right and B) left knee. Peptide injection was compared to CIA.

FIG. 18 shows Global histological score of right and left knees after S.C. injection. Peptide injection was compared to CIA (KW p<0.0001; p values are given versus respective controls).

FIG. 19 consists of FIGS. 19A, 19B, 19C, and 19D which show detailed criteria of evaluation of the histological score after S.C. injection. Peptide injection was compared to CIA. A) Inflammation (0-6) (KW, p<0.0001); B) Cartilage matrix proteoglycan loss (0-3) (KW; p<0.0001); C) Cartilage degradation (0-3) (KW, p<0.0001); and D) subchondral bone modification (0-3) (KW, p<0.0001). P values on the graphs are given versus respective controls.

FIG. 20 shows Arthritis visual score after I.A. injection. Peptide injection was compared to SCW.

FIG. 21 consists of FIGS. 21A and 21B which show Knee diameter measured after I.A. injection. Peptide injection was compared to SCW.

FIG. 22 consists of FIGS. 22A and 22B which show global histological score of right (A; KW, p<0.0001) and left (B; KW, p=0.0003) knees after I.A. injection. Peptide injection was compared to SCW. P values on the graphs are given versus respective controls except otherwise specified.

FIG. 23 consists of FIGS. 23A, 23B, 23C, and 23D which show detailed criteria of evaluation for the right knee of the histological score after I.A. injection. Peptide injection was compared to SCW. A) Inflammation (0-6) (KW, p<0.0001); B) Cartilage matrix proteoglycan loss (0-3) (KW; p=0.0145); C) Cartilage degradation (0-3) (KW, p=0.0004); and D) subchondral bone modification (0-3) (KW, p=0.0038). P values on the graphs are given versus respective controls.

FIG. 24 consists of FIGS. 24A, 24B, 24C, and 24D which show detailed criteria of evaluation for the left knee of the histological score after I.A. injection. Peptide injection was compared to SCW. A) Inflammation (0-6) (KW, p=0.0026); B) Cartilage matrix proteoglycan loss (0-3) (KW; p=0.0016); C) Cartilage degradation (0-3) (KW, p=0.0009); and D) subchondral bone modification (0-3) (KW, p=0.0015). P values on the graphs are given versus respective controls.

### DETAILED DESCRIPTION OF THE INVENTION

The present inventors found that Coll2-1 and Coll2-1NO₂ peptides have pro-infammatory, pro-angiogenic and immunomodulatory properties directly related with osteoarthritis (OA) and rheumatoid arthritis. These results demonstrate that Coll2-1, a marker of cartilage degradation is also involved in the physiopathology of OA. Therefore, a decrease of Coll2-1 release or its neutralization will have therapeutic effect by decreasing local and systemic inflammatory and immune responses. Therefore, the present invention provides means to alleviate or reduce or prevent the effects of Coll2-1 peptide and of Coll2-1NO₂ peptide and to treat and/or prevent osteoarthritis.

The nitrated from of the peptide is also relevant for the present invention. Peptide nitration is mainly caused by interaction of aromatic amino acids with peroxynitrite anion (ONOO⁻), a strong oxidant formed by the reaction of nitric oxide (^{▪}NO) and superoxide anion (O₂⁻^{^{▪}}). Tyrosine, phenylalanine and tryptophan residues are particularly sensitive to nitration. As demonstrated for type I collagen, type II collagen is susceptible of nitration by peroxynitrite. High levels of nitrite/nitrate have been found in the serum and articular fluids of patients with OA and RA indicating that production of ONOO⁻ is increased in these diseases. Furthermore, chondrocytes can produce both O₂⁻^{^{▪}} and ^{▪}NO and nitrotyrosine has been found in cartilage of arthritic patients. It has also been reported that N-iminoethyl-L-lysine, a selective inhibitor of the inducible nitric oxide synthase, reduces the progression of experimental OA induced in dog. Altogether, these findings indicate that ^{▪}NO or derived reactive oxygen species play a major role in the structural changes in arthritis and suggest that cartilage matrix components can be nitrated in situ and thereafter released in the synovial fluid.

Thus, the present invention provides a medicament comprising as active ingredient an inhibitor of Coll2-1 peptide activity and/or an inhibitor of Coll2-1NO₂ peptide activity for use in the prevention and/or treatment of osteoarthritis.

As used herein, an inhibitor of Coll2-1 peptide activity and/or an inhibitor of Coll2-1NO₂ peptide activity preferably is an agent which selectively decreases or blocks the activity of the respective peptide, in particular the activity on H₂O₂ production, on expression of angiogenic factors and/or on expression of IL-6 and IL-8 in osteoarthritic synovial fibroblast cells.

In respect to the angiogenic factors the inhibitor of Coll2-1 or Coll2-1 NO₂ peptide activity inhibits or reduces the enhancing effects of Coll2-1 or Coll2-1NO₂ peptide activity on expression of pro-angiogenic factors (VEGF). Further, the inhibitor of Coll2-1 or Coll2-1NO₂ peptide activity inhibits or reduces the reducing effects of Coll2-1 or Coll2-1NO₂ peptide activity on expression of anti-angiogenic factors (TSP1).

In particular, the present invention provides a medicament comprising an immunological binding partner which is immune-reactive with an epitope comprised in the amino acid sequence HRGYPGLDG (SEQ ID NO: 1), preferably the immunological binding partner is selected from the group consisting of polyclonal antibodies, monoclonal antibodies, humanized antibodies, Fc fragments, Fab fragments, single chain antibodies (scFv), chimeric antibodies, biobetters or other antigen-specific antibody fragments specifically binding to Coll2-1 or Coll2-1NO₂ peptide.

In a particularly preferred embodiment the immunological binding partner is monoclonal or polyclonal antibody specifically binding to Coll2-1 or Coll2-1NO₂ peptide.

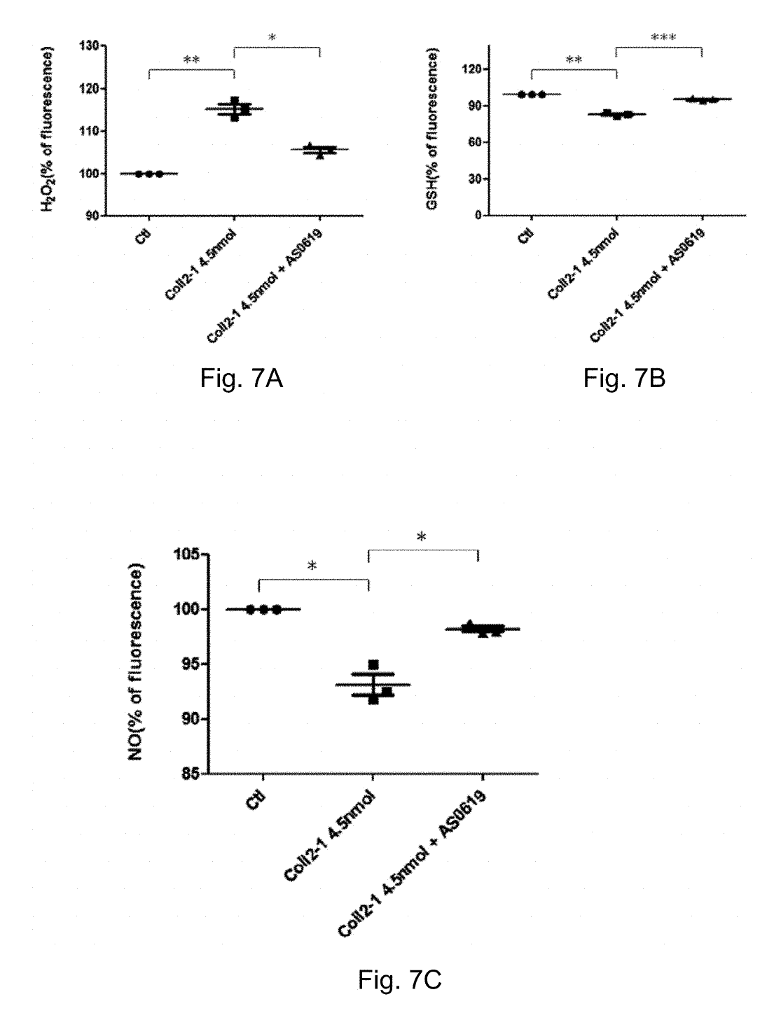

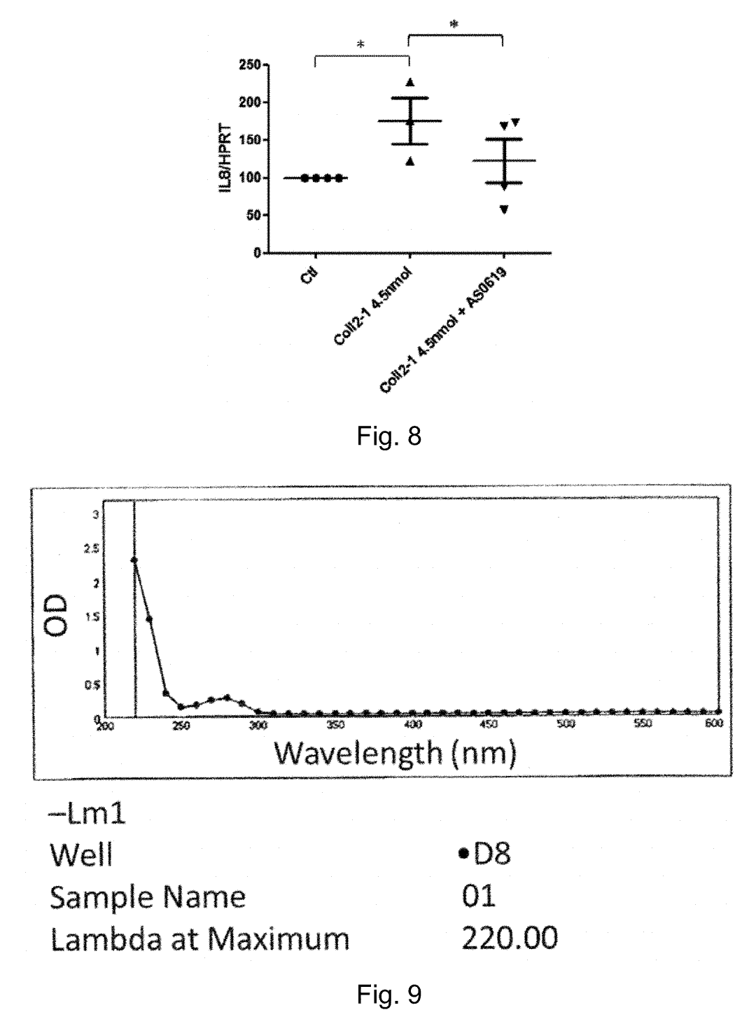

By providing such inhibitors in the medicament the Coll2-1 or Coll2-1NO₂ peptide activity on the H₂O₂ production, on expression of angiogenic factors and/or on expression of IL-6 and IL-8 in osteoarthritic synovial fibroblast cells is reduced or prevented. A polyclonal antibody (AS0619) prepared during the studies for the present invention reduced or neutralized the effect of the Coll2-1 peptide on the oxidative stress parameters and IL8 gene expression. As illustrated in FIG. 7, the polyclonal antibody AS0619 reversed significantly the effects of Coll2-1 peptide on the intracellular production of H₂O₂ (FIG. 7A) and GSH (FIG. 7B), on the production of NO (FIG. 7C) and the IL8 expression (FIG. 8). Most particularly, the polyclonal antibody AS0619 significantly decreased the IL8 expression in the OA synovial fibroblasts cells.

As mentioned above, said inhibitor of Coll2-1 or Coll2-1NO₂ peptide activity is an immunological binding partner which is immune-reactive with an epitope comprised in the amino acid sequence HRGYPGLDG.

This immunological binding partner which is immune-reactive with an epitope comprised in the amino acid sequence HRGYPGLDG, i.e. the monoclonal or polyclonal antibody specifically binding to Coll2-1 or Coll2-1NO₂ peptide was already described in WO03/076947. There, the antibody was described to be useful in the detection of the collagen type II derived sequence HRGYPGLDG.

### Antiserum Specificity

WO03/076947 describes that two antisera, D3 and D37, with a high specificity for Coll2-1 and Coll2-1NO₂, respectively, were identified. Antiserum D3 did not recognize human native type I and II collagens, human heat denatured type I and II collagens, and BSA. This suggested that antiserum D3 was specific for the linear form of Coll2-1. D3 also recognized, with the same affinity, the nitrated form of Coll2-1. Antiserum D37 showed a high affinity for Coll2-1NO₂. Thus, the cross-reactivity of non-nitrated peptide (Coll2-1) and human nitrated type II collagen with antiserum D37 was calculated to 0.02% and less than 0.08%, respectively. Furthermore, antiserum D37 did not recognize human nitrated type I collagen, native human type I and II collagens, nitrated BSA, BSA and 3-nitro-L-tyrosine residue. The very high concentrations of Coll2-1 and nitrated collagen type II needed to displace the Coll2-1NO2/D37 binding suggested that D37 was specific for Coll2-1NO₂.

As used herein, “immunological binding partner” includes polyclonal, monoclonal or humanized antibodies, including Fc fragments, Fab fragments, chimeric antibodies, biobetters or other antigen-specific antibody derivatives such as single chain antibodies scFvs.

The Coll2-1 peptide contains all of the following sequence HRGYPGLDG. The HRGYPGLDG sequence is unique for the collagen type II chain and is located in the helical part of collagen type II (position 289-297 GeneBank accession No. NP-001835 isoform 1 and position 220-228 GeneBank accession No. Nu-149162 isoform 2).

Antibodies with properties as described here, have been be raised against a synthetic peptide constituting the HRGYPGLDG sequence or another suitable protein or peptide fragment containing this sequence. Such an antibody possess reactivity toward collagen type II protein or fragments thereof from any species containing this epitope, among these are cow, dog, mouse, human, horse and rat. The peptide has been used as an antigen for immunization. The peptide is emulsified in an adjuvant medium, preferably incomplete Freund's adjuvant and injected subcutaneously or into the peritoneal cavity of a mammalian host, preferably a rodent most preferred rabbits, even more preferred mice. To enhance immunogenic properties of the antigenic peptide, it can be coupled to a carrier protein before emulsified in an adjuvant medium. Useful carriers are proteins such as keyhole limpet hemocyanin (KLH), edestin, albumins, such as bovine or human serum albumin (BSA or HSA), tetanus toxoid, and cholera toxoid, polyamino acids, such as poly- (D-lysine-D-glutamic acid). Booster injections may be given at regular intervals until an immune response is obtained, the last injection may be given intravenously to ensure maximal B-cell stimulation.

Antisera were screened for their ability to bind an epitope within the HRGYPGLDG sequence. Monoclonal antibodies were generated from immunized mice with the most promising antibody titer, by fusing lymphocytes isolated from the spleen of these mice with a myeloma cell line. The generated hybridoma clones were screened for antibodies with reactivity toward an epitope within the HRGYPGLDG sequence, and cell lines were be established for production and purification of monoclonal antibodies.

Methods for polyclonal and monoclonal antibody production and screening are well known in the art and other methods than the described can also be utilized.

### Formulations and Pharmaceutical Compositions

The following description refers to pharmaceutical compositions which may contain one or more active agents of the present invention.

The compositions of the invention will be formulated for administration through ways known in the art and acceptable for administration to a mammalian subject, preferably a human. In some embodiments of the invention, the compositions of the invention can be administered by oral, intravenous, intraperitoneal, intramuscular, transdermal, nasal, iontophoretic, subcutaneous, intratumoral, administration or by any other acceptable route of administration. In further embodiments of the invention the compositions of the invention are administered “locoregionally”, i.e., intravesically, intralesionally, and/or topically. In preferred embodiments of the invention, the compositions of the invention are administered systemically by injection, inhalation, suppository, transdermal delivery, etc. In further embodiments of the invention, the compositions are administered through catheters or other devices to allow access to a remote tissue of interest, such as an internal organ. The compositions of the invention can also be administered in depot type devices, implants, or encapsulated formulations to allow slow or sustained release of the compositions.

In order to administer therapeutic agents based on, or derived from, the present invention, it will be appreciated that suitable carriers, excipients, and other agents may be incorporated into the formulations to provide improved transfer, delivery, tolerance, and the like.

A multitude of appropriate formulations can be found in the formulary known to all pharmaceutical chemists: Remington's Pharmaceutical Sciences, (15th Edition, Mack Publishing Company, Easton, Pa. (1975)), particularly Chapter 87, by Blaug, Seymour, therein. These formulations include for example, powders, pastes, ointments, jelly, waxes, oils, lipids, anhydrous absorption bases, oil-in-water or water-in-oil emulsions, emulsions carbowax (polyethylene glycols of a variety of molecular weights), semi-solid gels, and semi-solid mixtures containing carbowax.

Any of the foregoing formulations may be appropriate in treatments and therapies in accordance with the present invention, provided that the active agent in the formulation is not inactivated by the formulation and the formulation is physiologically compatible.

The quantities of active ingredient necessary for effective therapy will depend on many different factors, including means of administration, target site, physiological state of the patient, and other medicaments administered. Thus, treatment dosages should be titrated to optimize safety and efficacy. Typically, dosages used in vitro may provide useful guidance in the amounts useful for in situ administration of the active ingredients. Animal testing of effective doses for treatment of particular disorders will provide further predictive indication of human dosage. Various considerations are described, for example, in Goodman and Gilman's the Pharmacological Basis of Therapeutics, 7th Edition (1985), MacMillan Publishing Company, New York, and Remington's Pharmaceutical Sciences 18th Edition, (1990) Mack Publishing Co, Easton Pa. Methods for administration are discussed therein, including oral, intravenous, intraperitoneal, intramuscular, transdermal, nasal, iontophoretic administration, and the like.

The compositions of the invention may be administered in a variety of unit dosage forms depending on the method of administration. For example, unit dosage forms suitable for oral administration include solid dosage forms such as powder, tablets, pills, capsules, and dragees, and liquid dosage forms, such as elixirs, syrups, and suspensions. The active ingredients may also be administered parenterally in sterile liquid dosage forms. Gelatin capsules contain the active ingredient and as inactive ingredients powdered carriers, such as glucose, lactose, sucrose, mannitol, starch, cellulose or cellulose derivatives, magnesium stearate, stearic acid, sodium saccharin, talcum, magnesium carbonate and the like. Examples of additional inactive ingredients that may be added to provide desirable color, taste, stability, buffering capacity, dispersion or other known desirable features are red iron oxide, silica gel, sodium lauryl sulfate, titanium dioxide, edible white ink and the like. Similar diluents can be used to make compressed tablets. Both tablets and capsules can be manufactured as sustained release products to provide for continuous release of medication over a period of hours. Compressed tablets can be sugar-coated or film-coated to mask any unpleasant taste and protect the tablet from the atmosphere, or enteric-coated for selective disintegration in the gastrointestinal tract. Liquid dosage is forms for oral administration can contain coloring and flavoring to increase patient acceptance.

The concentration of the compositions of the invention in the pharmaceutical formulations can vary widely, i.e., from less than about 0.1%, usually at or at least about 2% to as much as 20% to 50% or more by weight, and will be selected primarily by fluid volumes, viscosities, etc., in accordance with the particular mode of administration selected.

The compositions of the invention may be administered by use of solid compositions. For solid compositions, conventional nontoxic solid carriers may be used which include, for example, pharmaceutical grades of mannitol, lactose, starch, magnesium stearate, sodium saccharin, talcum, cellulose, glucose, sucrose, magnesium carbonate, and the like. For oral administration, a pharmaceutically acceptable nontoxic composition is formed by incorporating any of the normally employed excipients, such as those carriers previously listed, and generally 10-95% of active ingredient, that is, one or more compositions of the invention, and more preferably at a concentration of 25%-75%.

For aerosol administration, the compositions of the invention are preferably supplied in finely divided form along with a surfactant and propellant. Typical percentages of compositions of the invention are 0.01%-20% by weight, preferably 1%-10%, The surfactant must, of course, be nontoxic, and preferably soluble in the propellant. Representative of such agents are the esters or partial esters of fatty acids containing from 6 to 22 carbon atoms, such as caproic, octanoic, lauric, palmitic, stearic, linoleic, linolenic, olesteric and oleic acids with an aliphatic polyhydric alcohol or its cyclic anhydride. Mixed esters, such as mixed or natural glycerides may be employed. The surfactant may constitute 0.1%-20% by weight of the composition, preferably 0.25%-5%. The balance of the composition is ordinarily propellant. A carrier can also be included, as desired, as with, e.g., lecithin for intranasal delivery.

The compositions of the invention can additionally be delivered in a depot-type system, an encapsulated form, or an implant by techniques well known in the art. Similarly, the compositions can be delivered via a pump to a tissue of interest.

The composition of the invention may also be provided in a kit as a slow-release composition such as a daily, weekly, monthly unit provided as a sponge, dermal patch, subcutaneous implant and the like in a wrapping or container. In this case, the patient may release a unit of the composition from the container and applies it as indicated in the kit instructions. The composition may then be replaced at the end of the specified period by a fresh unit, and so on.

The compound(s) of the present invention may be administered in a composition that also comprises one or more further drugs. The proportion of compounds of the present invention to the other drug(s) and carrier may be adjusted accordingly.

### Antibodies

The invention in particular refers to the antibodies as an antibody directed to Coll2-1 peptide or Coll2-1NO₂ peptide will partially or completely reduce the activity of this peptide. The present invention further provides compositions comprising antibodies that specifically bind to Coll2-1 or Coll2-1NO₂ peptide. The peptide to which the antibody specifically binds is having the amino acid sequence SEQ ID NO: 1. The antibodies may be polyclonal antibodies, monoclonal antibodies, humanized antibodies, Fc fragments, Fab fragments, single chain antibodies (scFv), chimeric antibodies or other antigen-specific antibody fragments. In particular, said antibody may be a common antibody (which is composed of two heavy protein chains and two light chains), Fab fragments of a common antibody, single-chain variable fragments or single-domain antibody (sdAb). The antibodies may be formulated with a pharmaceutically acceptable carrier. In a preferred embodiment the antibodies specifically recognize and bind to Coll2-1 or Coll2-1NO₂ having the amino acid sequence SEQ ID NO: 1. Further preferred the antibodies specifically recognize an epitope (a stretch of 5 or more consecutive amino acid residues within the amino acid sequence shown in SEQ ID NO: 1).

The term “antibody,” as used herein, refers to a full-length (i.e., naturally occurring or formed by normal immunoglobulin gene fragment recombinatorial processes) immunoglobulin molecule (e.g., an IgG antibody) or an immunologically active (i.e., specifically binding) portion of an immunoglobulin molecule, including an antibody fragment. “Antibody” and “immunoglobulin” are used synonymously herein. An antibody fragment is a portion of an antibody such as F(ab′)2, F(ab)2, Fab′, Fab, Fv, scFv, Nanobodies and the like. Nanobodies (or single-domain antibodies (sdAb)) are antibody-derived therapeutic proteins that contain the unique structural and functional properties of naturally-occurring heavy-chain antibodies. The Nanobody technology was originally developed following the discovery that camelidae (camels and lamas) possess fully functional antibodies that lack light chains. These heavy-chain antibodies contain a single variable domain (VHH) and two constant domains (C_{H}2 and C_{H}3). Importantly, the cloned and isolated VHH domain is a perfectly stable polypeptide harbouring the full antigen-binding capacity of the original heavy-chain antibody. The antibodies could be obtained using immunization in human and animals (mouse, rabbit, camel, lama, hen, goat).

The term “antibody,” as used herein, also includes biobetters or bio-better antibodies. Bio-better antibodies are antibodies that target the same validated epitope as a marketed antibody, but have been engineered to have improved properties, e.g., optimized glycosylation profiles to enhance effector functions or an engineered Fc domain to increase the serum half-life.

As mentioned above, the term “antibody” used in the present invention further also include peptides containing at least one antigen-binding site formed of the variable region in the H chain or L chain of the antibody, or a combination thereof, Fab composed of a set of an H chain fragment and an L chain fragment, F(ab′)2 composed of two sets of H chain fragments and L chain fragments, single chain antibodies composed of an H chain fragment and an L chain fragment bound in series in a single peptide (hereunder, may be referred to as “scFvs”), and the like. The “antibody” of the present invention may be a full length antibody in a form normally existing in vivo, which is composed of two sets of full length H chains and full length L chains.

is The terms “F(ab′)2” and “Fab” in the present invention mean antibody fragments produced by treatment of an immunoglobulin with a protease such as pepsin or papain, and generated by digestion around the disulfide bonds existing between two H chains in the hinge region. For example, when IgG1 is treated with papain, cleavages occur upstream of the disulfide bonds existing between two H chains in the hinge region to allow the production of two identical antibody fragments in which an L chain composed of VL (L chain variable region) and CL (L chain constant region) and an H chain fragment composed of VH (H chain variable region) and CHγ1 (γ1 region within H chain constant region) are connected by a disulfide bond at the C-terminal region. These two identical antibody fragments are respectively referred to as Fab′. In addition, when IgG is treated with pepsin, cleavages occur downstream of the disulfide bonds existing between two H chains in the hinge region to allow the production of an antibody fragment which is slightly larger than the combined product having said two Fab's connected by the hinge region. This antibody fragment is referred to as F(ab′)2.

Usually, an antibody consists of two types of large and small polypeptides. The large subunit is referred to as “H chain (heavy chain)” and the small subunit is referred to as “L chain (light chain)”. In addition, each peptide is composed of a “variable region” existing at the N-terminal side and forming an antigen-binding site, and a “constant region” which is conserved per each antibody class. The variable region is further divided into complementarity determining regions “CDRs” which particularly involve the formation of the antigen-binding site, and “framework regions” existing therebetween. CDRs are known consist of three regions called “CDR1”, “CDR2”, and “CDR3” from the N-terminal side, for each H chain and L chain.

The single chain antibodies which may be used in the medicament of the present invention can be prepared by appropriately selecting inducible vectors such as pSE380 plasmid (Invitrogen) or pET24d(+) plasmid (Novagen) and host bacterial cells. In addition to the above method, in the production of the antibodies used in the present invention, an animal cell expression system, an insect cell expression system, and a yeast cell expression system can also be used. The linker for linking the H chain and the L chain can also be appropriately selected by those skilled in the art.

Further, an antibody in a form normally existing in vivo can be prepared from scFv. For example, only the variable regions of the H chain and the L chain are amplified by PCR from a scFv plasmid. Each fragment is, for example, recombined into a plasmid having the H chain gene and/or the L chain gene of a human antibody, which thereby enables the formation of an antibody having a variable region on the scFv in a form normally existing in vivo. Specifically, for example, appropriate restriction enzyme cleavage sites are introduced at both ends of the gene fragment obtained when amplifying the variable regions of the H chain and the L chain from the plasmid, and they are combined with an appropriate restriction enzyme cleavage site on the plasmid having the H chain and/or the L chain of the human antibody, thereby replacing genes in the variable region without causing frame-shift. Thus, an antibody in a form normally existing in vivo which has a sequence of a variable region on a plasmid as it is, can be prepared. Further, a peptide containing at least one antigen-binding site formed of the variable region of the H chain or L chain of the antibody, or a combination thereof, Fab composed of a set of an H chain fragment and an L chain fragment, and (Fab′2) composed of two sets of H chain fragments and L chain fragments can also be prepared from the antibody.

The expression of the antibody of the present invention can be carried out by employing E. coli, yeast, insect cells, animal cells, and the like. For example, when an antibody is expressed in COS cell or CHO cell, pCDNA3.1(+) or pMAMneo (CLONETECH) can be used. For example, a gene of the H chain of the antibody obtained in the above method is incorporated into a multicloning site of pCDNA3.1(+), and a gene of the L chain is incorporated into pMAMneo. Then, an expression unit having a gene of the L chain between a promoter and poly A is incorporated into an adequate site of the vector having the H chain incorporated therein. Introduction of this vector into a COS cell, a CHO-K1, or a CHO DG44 by a conventional genetic engineering technique enables the production of the antibody of interest. Further, the expression unit of the DHFR gene is cleaved out from for example, pSV2/DHFR (Nature, 1981. Vol. 294, Lee F. et al.) into the above prepared vector, and is incorporated into a vector which expresses the H chain and the L chain. This vector is introduced into the CHO DG44 by a conventional genetic engineering technique. Thus selected cells can be used to significantly improve the productivity of antibodies by utilizing the DHFR gene amplification system using MTX.

Animal cells such as COS cell or CHO cell can be generally cultured in Dulbecco's Modified Eagle's Medium (DMEM) supplemented with 10% fetal bovine serum (FBS) under 5% CO₂ at 37° C. A method for introducing a gene into the COS cell may be electroporation, as well as a DEAE-dextran method and a method using a transfection reagent such as lipofectin.

At the time of production of the antibody used in the medicament of the present invention, the cells are preferably cultured in a serum-free medium in order to prevent the contamination of a serum-derived bovine antibody. COS and CHO cells which are not acclimatized in a serum-free medium but are cultured in serum media, are preferably cultured in serum-free DMEM. The antibody of the present invention, which is thus obtained in the culture supernatant, can be easily purified by a conventional method for purifying IgG antibodies using, for example, Protein A column and Protein G column.

Regardless of structure, an antibody fragment binds with the same antigen that is recognized by the full-length antibody, and, in the context of the present invention. Methods of making and screening antibody fragments are well-known in the art.

An anti-Coll2-1 antibody according to the present invention may be prepared by a number of different methods. For example, the antibodies may be obtained from subjects administered the recombinant polypeptide or peptide HRGYPGLDG. In some embodiments, the antibodies may be made by recombinant methods. Techniques for making recombinant monoclonal antibodies are well-known in the art. Recombinant polyclonal antibodies can be produced by methods analogous to those described in U.S. Patent Application 2002/0009453, using the recombinant polypeptide according to the present invention as the immunogen (s). Said antibody obtained in accordance with the invention may be a murine, human or humanized antibody. A humanized antibody is a recombinant protein in which the CDRs of an antibody from one species; e.g., a rodent, rabbit, dog, goat, horse, camel, lama or chicken antibody (or any other suitable animal antibody), are transferred from the heavy and light variable chains of the rodent antibody into human heavy and light variable domains. The constant domains of the antibody molecule are derived from those of a human antibody. Methods for making humanized antibodies are well known in the art. More recently, it was reported that it is possible to generate hybridomas directly from human B-cells. Consequently, the recombinant polypeptide or peptide HRGYPGLDG could be used to stimulate proliferation of human B-cell before to proceed to the generation of hybridomas.

The above-described antibodies can be obtained by conventional methods. For example, the recombinant polypeptide according to the present invention can be administered to a subject and the resulting IgGs can be purified from plasma harvested from the subject by standard methodology.

### Antibody Compositions

The invention also refers to the preparation of antibodies and antibody compositions suitable for administration, such as compositions comprising an antibody and a pharmaceutically acceptable carrier. The antibody compositions may be formulated for any route of administration, including intravenous, intramuscular, subcutaneous and percutaneous, by methods that are known in the art. In one embodiment, the antibody composition provides a therapeutically effective amount of antibody, i.e., an amount sufficient to achieve a therapeutically beneficial effect.

In one embodiment, the specific IVIG composition comprises both an antibody that specifically binds to Coll2-1 or Coll2-1NO₂ peptide. The antibodies and antigens may be any of those previously described.

Treatment of Osteoarthritis and Other Rheumatic and Musculoskeletal Diseases with Antibodies or Antibody Compositions

The present invention also refers to a method of treating osteoarthritis and other rheumatic and musculoskeletal diseases (RMDs) by administering the above-described antibody or antibody compositions, to a subject in need thereof. A target patient population for the treatment of osteoarthritis includes mammals, such as humans, who suffer of osteoarthritis.

In accordance with one embodiment, the invention provides a method for treating osteoarthritis using compositions comprising an antibody or antibodies directed to Coll2-1 or Coll2-1NO₂ according to the present invention, and a pharmaceutically acceptable carrier. The medicament for treating osteoarthritis comprises polyclonal antibodies, monoclonal antibodies, humanized antibodies, Fc fragments, Fab fragments, single chain antibodies (scFv), chimeric antibodies or other antigen-specific antibody fragments specifically binding to Coll2-1 or Coll2-1NO₂ peptide. In yet another embodiment, the antibodies are monoclonal antibodies specifically binding to Coll2-1 or Coll2-1NO₂ peptide.

A therapeutically effective amount of the antibody compositions can be determined by methods that are routine in the art. Skilled artisans will recognize that the amount may vary according to the particular antibodies within the composition, the concentration of antibodies in the composition, the frequency of administration, the severity of disease to be treated, and subject details, such as age, weight and immune condition. A therapeutically effective amount will be from 10 ng to 100 mg/kg of body weight. In some embodiments the dosage will be at least about 0.5 mg/kg at least about 1 mg/kg, at least about 5 mg/kg, at least about 10 mg/kg, at least about 15 mg/kg, at least about 20 mg/kg, or at least about 25 mg/kg. The route of administration may be any of those appropriate for a passive vaccine. Thus, intravenous, subcutaneous, intramuscular, intraperitoneal, intratumorally and other routes of administration are envisioned. As noted above, a therapeutically effective amount of antibody is an amount sufficient to achieve a therapeutically beneficial effect. A protective antibody composition may decrease tumor size and prevent spreading and metastasis.

The antibody composition may be administered in conjunction with an anti-osteoarthritis agent. The anti-osteoarthritis agents may be combined prior to administration, or administered concurrently or sequentially with the antibody or antibody composition.

### EXAMPLES

### Example 1: Patients and Methods

1.1 Patients. Synovial tissue samples were obtained from 10 patients (8 women, 2 man; mean age 70 +/−6 years) with osteoarthritis (OA) of the knee at the time of total knee joint replacement surgery. All the subjects provided their informed consent.

1.2 Isolation and culture of synovial cells. Synovium biopsies were digested is with collagenase from Clostridium histolyticum, Type IA (1 mg/ml) (Sigma-Aldrich, Saint-Louis, USA) and 0.25% trypsin (Sigma Aldrich, Saint Louis, USA) with 0.68 mM EDTA in Tris buffer 0.1M, pH 7.4 in complete medium (CM) (DMEM (Dulbecco's Modified Eagle's Medium) Low Glucose supplemented with 10 mM HEPES, 100 U/ml penicillin, 100 μg/ml streptomycin, 2 mM glutamine and 10% fetal calf serum (Lonza, Verviers, Belgium)) for 1 hour at 37° C. The cells suspensions were passed through a 70-μm filter to remove any undigested tissue. The filtered cells suspensions were then collected by centrifugation at 800 g and were cultured in culture flasks. After 24 hours, medium was changed to eliminate non adherent cells. Synovial fibroblasts cells (SFC) were used after three passages.

1.3 Treatment of synovial fibroblast cells. Cells were seeded into a six-well plate at the density of 2×10⁵ cells/well in 2 ml of CM. When the cells reached confluence, the CM was replaced with 1% serum medium for 24 h to keep the cells quiescent. Cells were incubated for 24 hours with or without peptides Coll2-1 (¹⁰⁸HRGYPGLDG¹¹⁶; 0.45 and 4.5 nmol) or Coll2-1NO₂ (¹⁰⁸HRGYPGLDG-NO₂¹¹⁶; 4 and 40 pmol) at a range of concentrations found in the blood of osteoarthritic patients.

1.4 RNA extraction and Real Time Reverse Transcriptase-Polymerase Chain Reaction (RT PCR). Total RNA were extracted using the RNeasy Mini Kit (Qiagen, Netherlands) and were reverse transcribed with SuperScript III Reverse Transcriptase (Invitrogen, Belgium) according to the manufacturer's instructions. The cDNAs were quantified by real-time quantitative PCR using the Rotor Gene instrument (Qiagen, Venlo, Netherlands) and the SYBR Premix Ex Taq kit (Takara, Verviers, Belgium). The level of gene expression was determined by interpolation with a standard curve. To standardize mRNA levels, HPRT, a house keeping gene, was amplified as an internal control. The oligonucleotide primers sequences were as follow: HPRT forward, 5′-TGTAATGACCAGTCAACAGGG-3′ (SEQ ID NO: 2); HPRT reverse, 5′-TGCCTGACCAAGGAAAGC-3′ (SEQ ID NO: 3); TSP-1 forward, 5′-CAGACCGCATTGGAGATAC-3′ (SEQ ID NO: 4); TSP-1 reverse, 5′-CCATCGTTGTCATCATCGTG-3′ (SEQ ID NO: 5); VEGF forward, 5′-TGCCTTGCTGCTCTAC-3′ (SEQ ID NO: 6); VEGF reverse, 5′-CACACAGGATGGCTTGAA-3′ (SEQ ID NO: 7); IL-6 forward, 5′-AAA CAA ATT CGG TAC ATC CTC G-3′ (SEQ ID NO: 8); IL-6 reverse, 5′-CCA GGC AAG TCT CCT CAT-3′ (SEQ ID NO: 9); IL-8 forward, 5′-GGA ACC ATC TCA CTG TGT GTA A-3′ (SEQ ID NO: 10); IL-8 reverse, 5′-TGG AAA GGT TTG GAG TAT GTC T-3′ (SEQ ID NO: 11).

1.5 Intracellular H₂O₂ and O₂^{∘}^{_{−}} productions. Levels of intracellular superoxide anion (O₂^{∘}^{_{−}}) and hydrogen peroxide (H₂O₂) were assessed spectrofluorimetrically by oxidation of specific probes: dihydroethidium (Molecular Probes, Leiden, The Netherlands) and H2DCFH-DA (Molecular Probes, Leiden, The Netherlands) respectively. Synovial cells seeded on 96-well plates (8×10³ cells per well) were washed once with phosphate-buffered saline and then incubated with 50 μL of phosphate-buffered saline containing either 250 μM dihydroethidium or 200 μM H2DCFH-DA. The fluorescence intensity was measured every hour for 6 hours. The levels of O₂^{∘}^{_{−}} or H₂O₂ are expressed in arbitrary unit/living cell. The numbers of adherent cells were measured by crystal violet assay.

1.6 GSH assay. Levels of intracellular GSH were assessed by monoclorobimane staining. Synovial cells seeded on 96-well plates (8×10³ cells per well) were washed once with phosphate-buffered saline and then incubated with 50 μM monoclorobimane diluted in phosphate-buffered saline. The fluorescence intensities were measured at 3° C. using excitation and emission wavelengths of 380 and 485 nm, respectively. The intracellular GSH level was expressed as arbitrary units of fluorescence intensity.

1.7 NO production. Nitric oxide production was evaluated using a DAF2-DA fluorescent probe (Molecular Probes, Leiden, The Netherlands). Cells Synovial cells seeded on 96-well plates (8×10³ cells per well) were washed once with phosphate-buffered saline and then incubated with 50 μL of 200 μM DAF2-DA solution. The fluorescence intensity was measured every hour for 6 hours. The levels of NO are expressed in arbitrary unit/living cell. The numbers of adherent cells were measured by cristal violet assay.

1.8 Antiserum AS0619. A polyclonal antibody was obtained after immunization of rabbits with the sequence Coll2-1 peptide (¹⁰⁸HRGYPGLDG¹¹⁶). Serum was purified on Protein A Columns (Pierce, Gent, Belgium) to retain only the serum IgG. The absorbance of the IgG at 280 nm was 1.974 (FIG. 9) and its concentration was 2.6649 mg/ml (1.974× molar extinction coefficient (IgG=1.35)).

1.9 Competitive inhibition of Coll2-1 peptide with an antiserum AS0619. Before to be added to synoviocytes culture, the Coll2-1 peptides were pre-incubated overnight at 4° C. under constant agitation with purified polyclonal antibody AS0619 (polyclonal antibody binding specifically Coll2-1 peptide) in Low Glucose DMEM supplemented with 10 mM HEPES, 100 U/ml penicillin, 100 μg/ml streptomycin, 2 mM glutamine The volume of this solution was of that which was required for the treatment of synovial fibroblast cells.

1.10 Statistical analysis. Data were analysed and compared using a Kruskal-Wallis test followed, if positive, by the Dunn's multiple comparison post-test. P-values were considered significant when P<0.05. All data were analysed using GraphPad Prism software V5.0.

### Example 2: Effects of Peptides Coll2-1 and Coll2-1NO₂ on Oxidative Stress Parameters in OA Synovial Fibroblast Cells

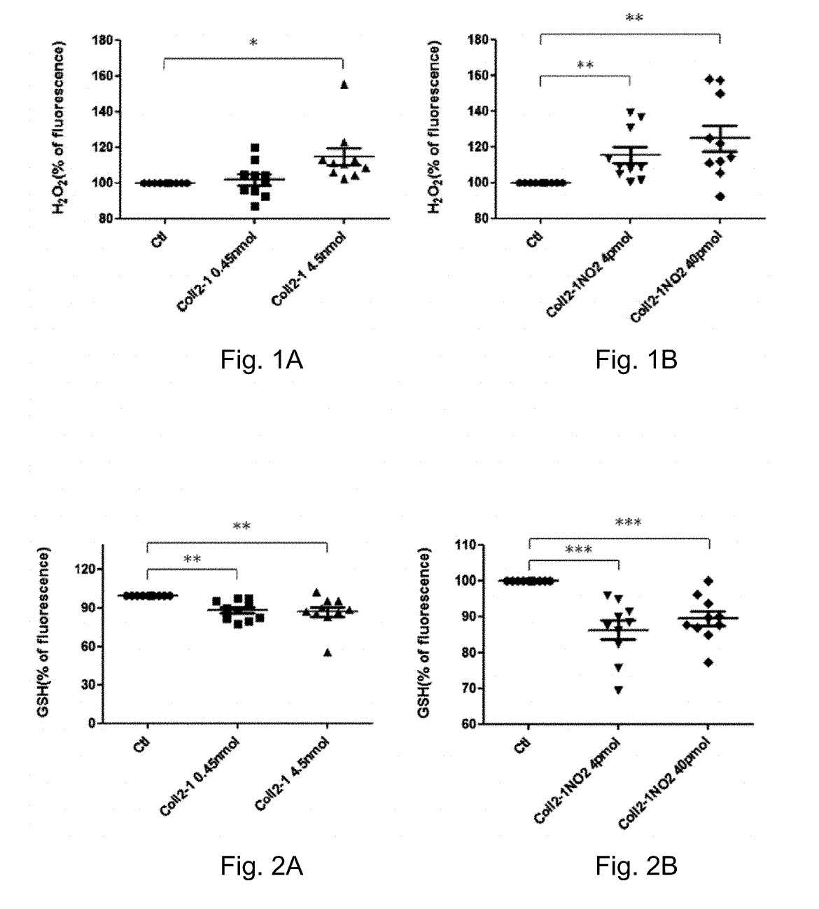

Using fluorescent probes, the effects of Coll2-1 and Coll2-1NO₂ peptides on the oxidative stress was investigated. To do this, several parameters were studied. The first outcome was the intracellular production of H₂O₂ by synovial fibroblast cells. As illustrated in FIGS. 1A and 1B, the intracellular production of H₂O₂ with both Coll2-1 (0.45 and 4.5 nmol) and Coll2-1NO₂ (4 and 40 pmol) peptides was significantly increased by Coll2-1 at the concentration of 4.5 nmol (*P<0.05) and by Coll2-1NO2 at the concentrations of 4 pmol (**P<0.01) and 40 pmol (**P<0.01).

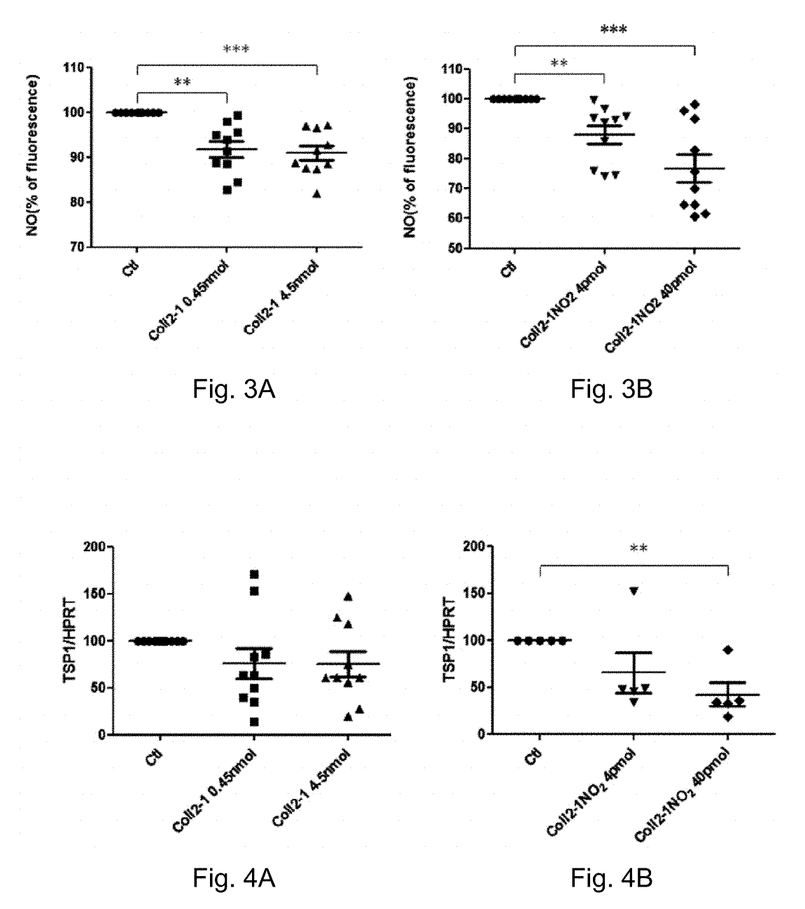

In parallel, the effects of these peptides on the intracellular level of the reduced form of glutathione, GSH, was evaluated. In the presence of Coll2-1 (at 0.45 and 4.5 nmol; **P<0.01) and Coll2-1NO₂ (at 4 and 40 pmol; ***P<0.001), a significant decrease of GSH was observed (FIGS. 2A and 2B). Finally, Coll2-1 and Coll2-1NO₂ peptides is also decreased significantly the production of NO (nitric oxide) in synovial fibroblast cells (FIGS. 3A and 3B). This effect was significantly important when cells were treated with Coll2-1 NO₂ (FIG. 3B).

### Example 3: Effects of Peptides Coll2-1 and Coll2-1NO₂ on Angiogenic Factors Expression in OA Synovial Fibroblast Cells

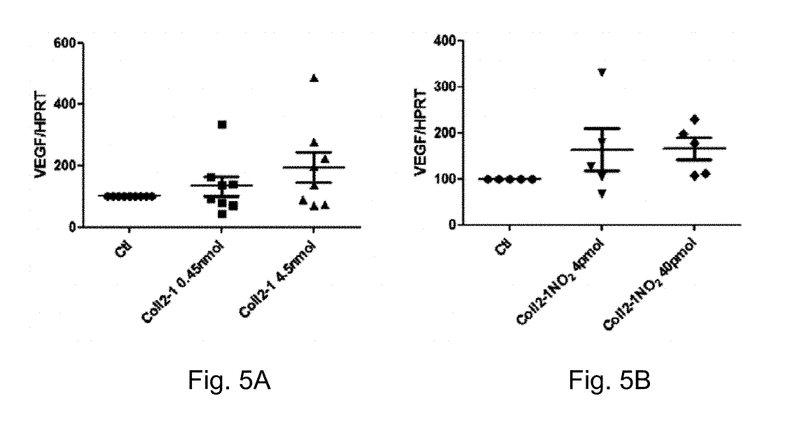

Using Real Time FOR technology, the effects of Coll2-1 and Coll2-1NO₂ peptides on the expression of angiogenic factors was investigated. Synovial fibroblast cells were incubated during 24 hours without or with increasing concentrations of Coll2-1 (0.45 and 4.5 nmol) or Coll2-1NO₂ (4 and 40 pmol) peptides. The expression of TSP1 (Thrombospondin (TSP)-1; anti-angiogenic factor) and VEGF (Vascular Endothelial Growth Factor; pro-angiogenic factor) gene was analyzed. As illustrated in FIG. 4, both Coll2-1 and Coll2-1NO₂ peptides decreased TSP1 expression. This effect was significant with Coll2-1NO2 at a concentration of 40 pmol (**P<0.01; FIG. 4B). In contrast, the VEGF expression tended to increase in the presence of increasing concentrations of Coll2-1 and Coll2-1NO₂ peptides, as presented in FIG. 5. Interestingly, the differential expression of these two genes revealed an imbalance between pro- and anti-angiogenic factors in favour of angiogenesis process.

### Example 4: Effects of Peptides Coll2-1 and Coll2-1NO₂ on Inflammatory Mediators Expression in OA Synovial Fibroblast Cells

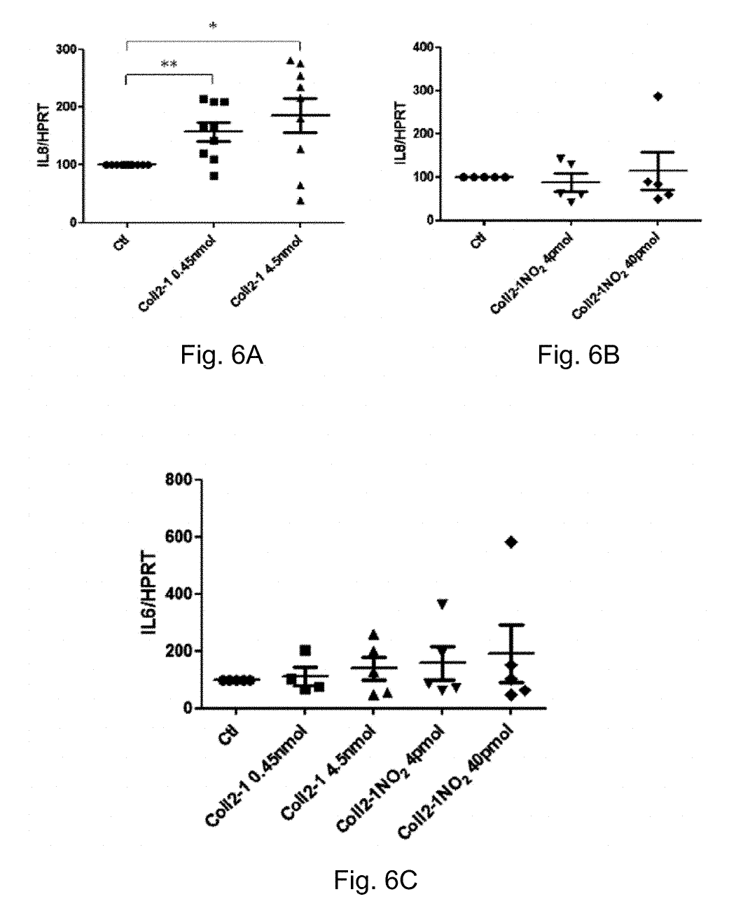

Further, the effects of Coll2-1 and Coll2-1NO₂ peptides on inflammatory process was also investigated. The expression of IL (interleukin)-8 and -6 genes, two genes implicated in the OA physiopathology process was analyzed. As shown in FIG. 6, the increasing concentrations of Coll2-1 and Coll2-1NO₂ peptides increased the expression of both cytokines by OA synovial fibroblast cells. Most particularly, Coll2-1 at 0.45 nmol (**P<0.01) and 4.5 nmol (*P<0.05) dose-dependently increased IL8 gene expression. These data highlight the pro-inflammatory properties of Coll2-1 peptides.

### Example 5: Competitive Inhibition of AS0619 with the Coll2-1 Peptide

To validate the hypothesis that antibodies or other agent neutralizing type II collagen peptides could be used as a biotherapy to treat arthritis, specific polyclonal antibodies directed to Coll2-1 peptide were tested. As described in Example 1 section 1.8 rabbits were immunized with Coll2-1 peptide (¹⁰⁸HRGYPGLDG¹¹⁶). A polyclonal antibody was obtained and purified on Protein A Columns (Pierce, Gent, Belgium) to retain only the serum IgG. The absorbance of the purified IgG was measured at 280 nm and was 1.974. The concentration was 2.6649 mg/ml (1.974×molar extinction coefficient (IgG=1.35)). The polyclonal antibody was named AS0619.

The effect of peptide neutralization on the oxidative stress parameters (FIG. 7) and IL8 gene expression (FIG. 8) by the polyclonal antibody AS0619 was examined. As illustrated in FIG. 7, AS0619 reversed significantly the effects of Coll2-1 peptide on the intracellular production of H₂O₂ (*P<0.05; FIG. 7A) and GSH (***P<0.001; FIG. 7B), on the production of NO (*P<0.05; FIG. 7C) and the IL8 expression (FIG. 8). Most particularly, AS0619 significantly decreased the IL8 expression (*P<0.05) in the OA synovial fibroblasts cells.

In the competition assay competetive inhibition of Coll2-1 peptide with the polyclonal antibody AS0619 was assayed. Before addition to synoviocytes culture, the Coll2-1 peptides were pre-incubated overnight at 4° C. under constant agitation with purified polyclonal antibody AS0619 in Low Glucose DMEM supplemented with 10 mM HEPES, 100 U/ml penicillin, 100 μg/ml streptomycin, 2 mM glutamine. The volume of this solution was ¼ of that which was required for the treatment of synovial fibroblast cells. As result, in the competition assay it was shown that the polyclonal antibody AS0619 at 1.33 μg/ml binds 500 nM of Coll2-1 peptide.

For the first time, it was shown that type II collagen peptides have pro-infammatory, pro-angiogenic and immunomodulatory properties directly related with osteoarthritis and rheumatoid arthritis. These results are critical. They demonstrate that Coll 2-1, a marker of cartilage degradation is also implicated in the physiopathology of OA. Therefore, a decrease of Coll 2-1 release or its neutralization with for example antibodies could have therapeutic effect by decreasing local and systemic inflammatory and inate immune responses. In this context, Coll 2-1 and its nitrated form represents a potential therapeutic target for a biotherapy.

### Example 6: Competitive Inhibition of D37 with the co112-1-NO2 Peptide

To validate the hypothesis that antibodies or other agent neutralizing type II collagen peptides could be used as a biotherapy to treat arthritis, we have tested a specific antiserum for Coll2-1NO₂ peptide. This antiserum was called D37.

### Methods:

Antiserum D37. A polyclonal antibody was obtained after immunization of rabbits with the sequence Coll2-1NO₂ peptide (¹⁰⁸HRGY(NO₂)PGLDG¹¹⁶).

Competitive inhibition of Coll2-1NO₂ peptide with an antiserum D37. Before to be added to synoviocytes culture, the Coll2-1NO₂ peptides were pre-incubated overnight at 4° C. under constant agitation with D37 (polyclonal antibody binding specifically Coll2-1NO₂ peptide) in Low Glucose DMEM supplemented with 10 mM HEPES, 100 U/ml penicillin, 100 μg/ml streptomycin, 2 mM glutamine. The volume of this solution was ¼ of that which was required for the treatment of synovial fibroblast cells.

### Results

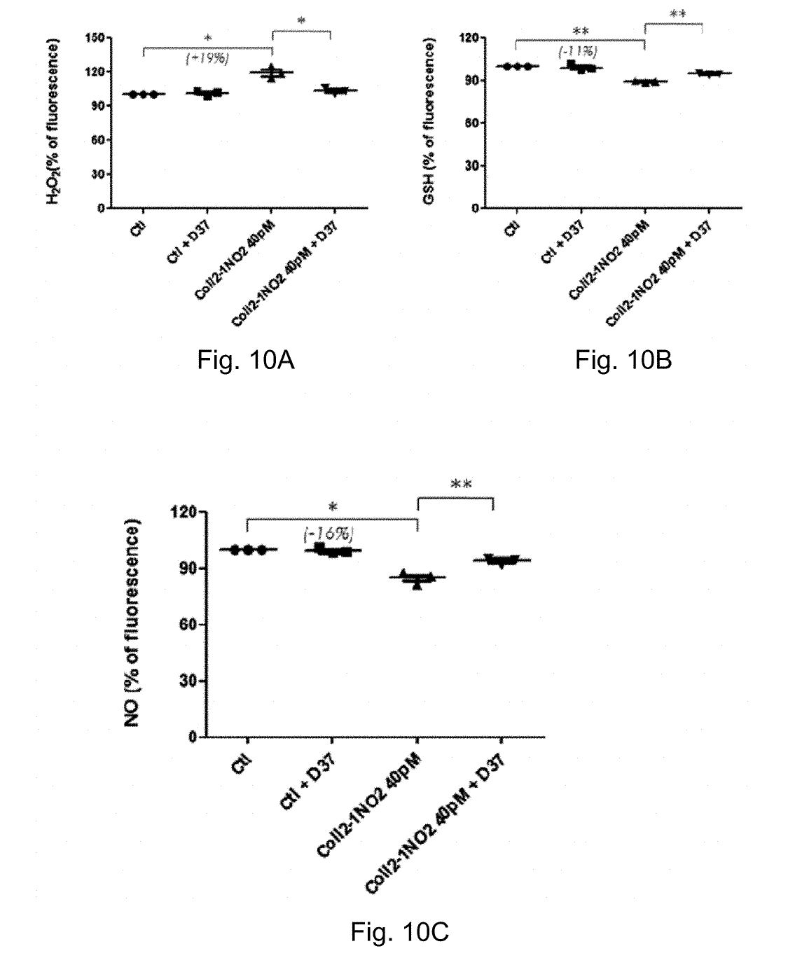

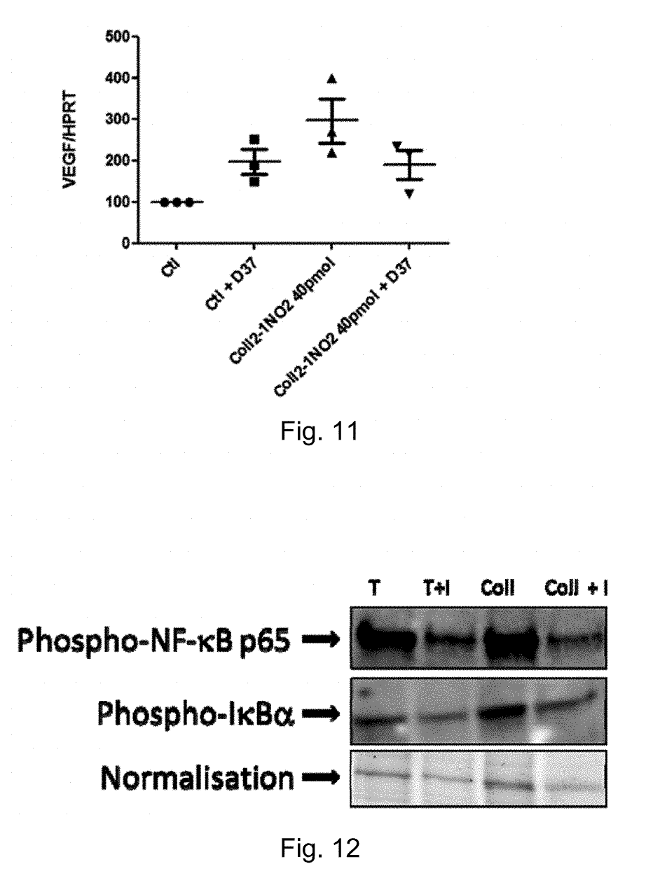

We examined the effect of peptide neutralization on the oxidative stress parameters (FIG. 10) and VEGF gene expression (FIG. 11). As illustrated in FIG. 10, D37 reversed significantly the effects of Coll2-1NO₂ peptide on the intracellular production of H₂O₂ (*P<0.05; FIG. 10A) and GSH (**P<0.01; FIG. 10B), on the production of NO (**P<0.01; FIG. 10C) and the VEGF expression (FIG. 11).

### Example 7 Effects of Coll2-1 on the NF-κB Activation

In the presence of Coll2-1, we observed the phosphorylation of p65 NF-κB subunit. Oxidative stress inhibitors (apocynin: 0.2 mM and diphenyleneiodonium: 6.35 10⁻² mM) inhibited Coll2-1 induced phosphorylation and translocation of p65. Further, to examine whether Coll2-1 induced NF-κB activation occurred through IκBα degradation, we probed the phosphorylation of IκBα subunit. Coll2-1 induced IκBα degradation and oxidative stress inhibitors decreased it (FIG. 12).

Western blotting. Cells were collected at 4° C. and lysed on ice in 50 μl of buffer (25 mM Hepes, 150 mM NaCl, 0.5% Triton X-100, 10% glycerol, and 1 mM dithiothreitol) containing protease and phosphatase inhibitors. After incubation at 4° C. for 30 minutes, lysates were then centrifugated at 14,000 g for 30 minutes at 4° C. to remove insoluble debris. Protein concentrations were determined using BCA assay. Total protein extracts (10 μg) were fractioned by electrophoresis on a polyacrylamide gel (10%) and transferred onto a PVDF membrane. Membrane were blocked for 1 h in TBS-Tween containing 2% bovine serum albumin (phosphor-NF-κB p65 and phospho-IκBα). Membranes were then incubated overnight at 4° C. with primary antibodies. Anti-rabbit phospho-NF-κB p65 (1:1000 dilution), anti-rabbit phospho-IκBα (1:1000 dilution) were used. Horse-radish peroxidase (HRP)-linked anti-rabbit IgG antibody (1:2000 dilution) was used as secondary antibody. The reaction was revealed with the ECL Western blotting substrate.

### Example 8 Inhibition of TLR-4 Receptor

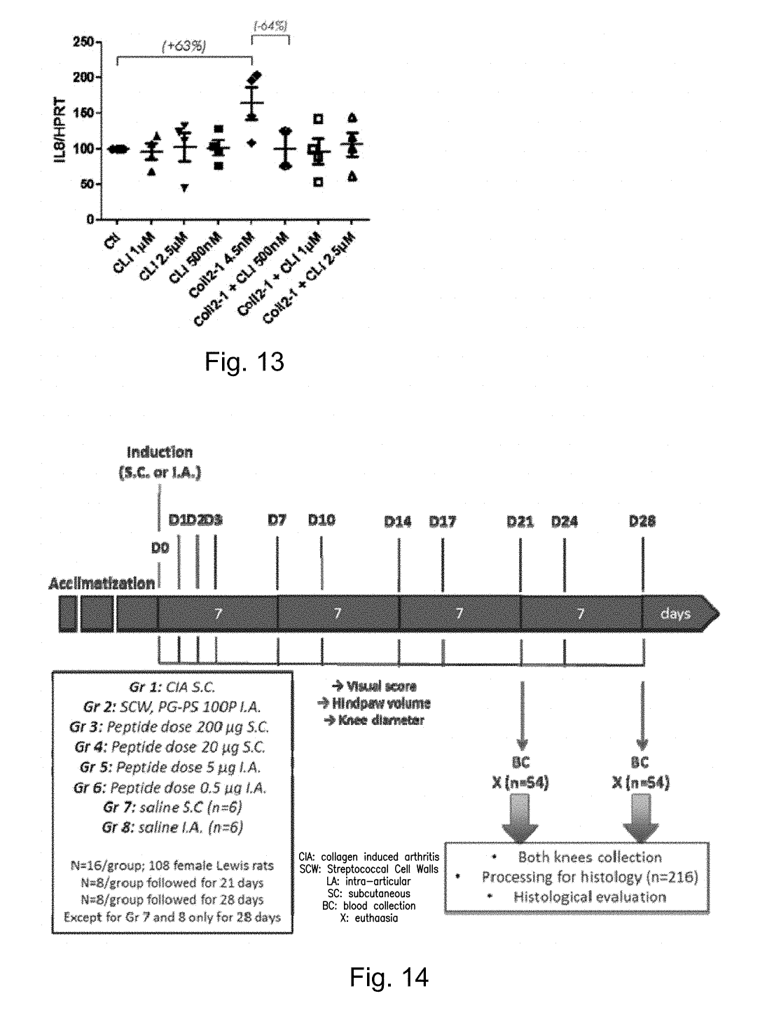

To identify the potential receptor of Coll2-1, we examined the effect of CLI-095, a TLR-4 signaling inhibitor. In the presence of the latter, we observed a decrease of IL-8 expression by osteoarthritic synoviocytes in the presence of Coll2-1 (FIG. 13).

Inhibition of TLR-4 receptor. Synoviocytes were pre-treated 1 hour with CLI-095 (500 nM, 1 and 2.5 μM) before a 24 hours treatment with Coll2-1 at 4.5 nmol.

### Example 9

An in-vivo study was performed to evaluate the potential immunizing effect of Coll2-1 peptide. This effect was evaluated in comparison with two validated models of rheumatoid arthritis: CIA (Collagen induced arthritis) and SCW (Streptococcal cell walls). The peptide has been injected once subcutaneous, at the base of the tail (S.C.—evaluation of systemic inflammation) or intra-articular (I.A.—evaluation of mono-articular inflammation) in female Lewis rats. Naïve controls (saline) were considered. Animals were euthanized 21 or 28 days after injection.

### Methods

9.1 Study design as illustrated in FIG. 14

All animals (n=108 females; 96.0-129.7 gr) included in the study were identified by a unique chip. They were randomly distributed into 8 study groups (n=16/group except nave controls n=6/group, n=8/each time point) before induction (DO) based on their body weight (FIG. 1).
Injected controls (2 groups)
Group 1: bovin type II collagen (CIA)—S.C. (n=16)
Group 2: streptococcal cell wail (SCW)—I.A. (n=16)
Test groups (4 groups)
Group 3: Peptide dose 200 μg/100 μl—S.C. (n=16)
Group 4: Peptide dose 20 μg/100 μl—S.C. (n=16)
Group 5: Peptide dose 5 μg/50 μl—I.A. (n=16)
Group 6: Peptide dose 0.5 μg/50 μl—I.A. (n=16)
Naïve controls (2 groups)
Group 7: saline S.C. (n=6)
Group 8: saline I.A. (n=6)

### 9.2 Method of Administration

Two injection ways were chosen to evaluate the potential effect of the peptide: intra-articular (I.A.) and subcutaneous (S.C.), two route classically used in the art.

Coll2-1 peptide was administered either I.A. under a 50 μL volume into the right knee or S.C. under a 100 μL volume at the base of the tail.

SCW was administered I.A. into the right knee under a 50 μL volume. Bovine type II collagen (CIA) was administered under 100 μL volume at the base of the tail.

### 9.3 Doses Levels

Two doses of the Coll2-1 peptide were tested in both conditions.

It was administered I.A. at 5 and 0.5 μg under 50 μL

It was administered S.C. at 200 and 20 μg under 100 μL.

Coll2-1 peptide was administered once (DO).

### 9.4 Parameters of Evaluation

9.4.1 Arthritis Visual Score

Joint was considered arthritic when significant signs of redness and/or swelling was noted. Each paw was evaluated with a visual score (0-3)
0: normal, no joint inflammation
1: large joint inflammation
2: medium joint inflammation
3: small joint (distal) inflammation

A set of 4 values were recorded on each evaluation day. The 4 values were summed for analysis.

9.4.2 Hind Paw Edema
Knee diameter

Right and left knees diameter were measured using a digital caliper.

### 9.5 Euthanasia

Ethical endpoints were:
Inflammation of the injected zone interfering with the normal behavior of the animal
General state of the animal altered (prostration, bristly hair, arched back, respiratory distress, bleeding or purulent flow, paralysis, abdominal distension, diarrheas) in spite of the implementation of adapted treatments (if possible)
Loss of more than 20% of the maximal weight or within 15 days.

Animals were euthanized 21 or 28 days after injection (D0). They were anesthetized with a subcutaneous injection of a Ketamin/Medetomidine mix and further euthanized by exsanguination.

### 9.6 Histological Evaluation

Both entire knees from each animal were processed for histological analysis, They were decalcified in a HCl/EDTA mix (DC2, Qpath, VWR Belgium) for the appropriate time before inclusion in paraffin. Three frontal sections (5 μm) of each compartment were done 200 μm apart in the weight bearing zone using a microtome. Slides were stained according to a standard Safranin-O/Fast green protocol.

Slides were evaluated under a light microscope. Each slide was score for:
Infiltration of inflammatory cells in synovial tissue (0-3)
Inflammatory cells in the joint cavity (exudate) (0-3)
Cartilage proteoglycan depletion (0-3)
Loss of articular cartilage (0-3)
Bone erosion (0-3).

Infiltration of inflammatory cells in synovial tissue and inflammatory cells in the joint cavity were considered together (sum of the scores) as inflammatory parameters for the analysis of the detailed criteria.

### 9.7 Statistical Analysis

Statistical analysis was performed with the appropriate test using GraphPad.
Visual score and evaluations (knee diameter measurements) were analyzed with a two way ANOVA followed, if positive, by Tukey's multiple comparison test. Graphs represented the mean of each group at each evaluated time point.
   Histological scores were analyzed with a Kruskal Wallis for non-parametric values followed, if positive, by a Dunn's multiple comparison test. Graphs presented as box and whisker plots. Exact p values were provided and asterisks representation were also performed.

### Results

### Body Weight

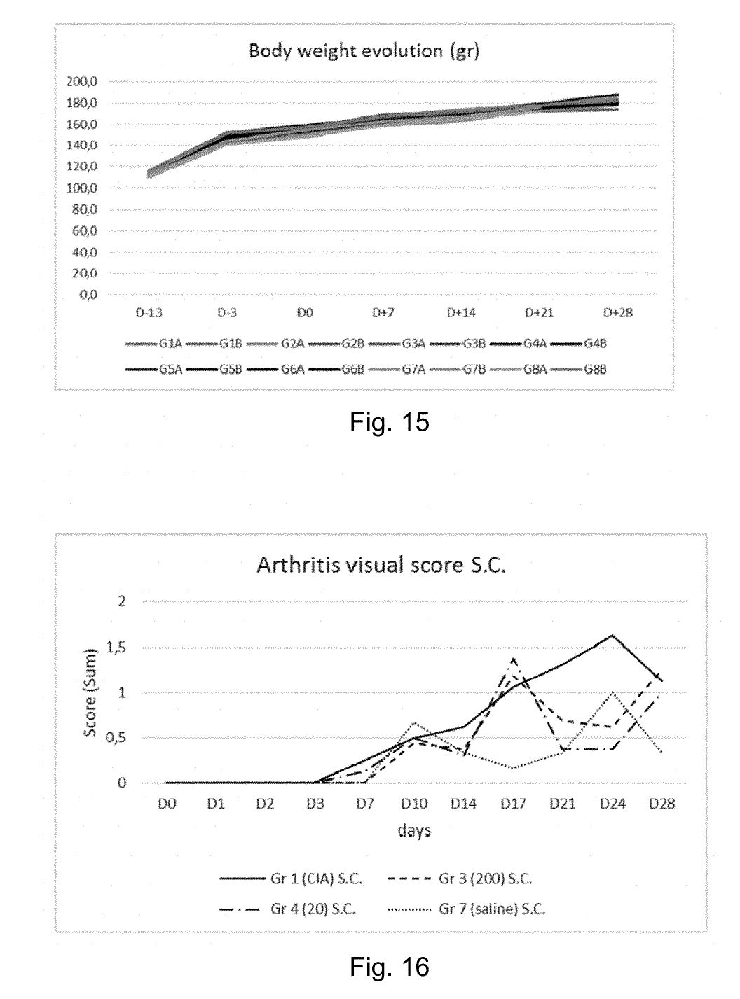

The body weight of the 8 groups was followed throughout the study as illustrated in FIG. 15). No lost of weight was observed for all the groups after the injection of the items. No specific difference was observed between the groups until the end of the study.

### Results According to CIA (Collagen Induced Arthritis) Model S.C. Injection

I. Arthritis score
   The injection of bovine type II collagen induced a reaction that was evaluated on the four limbs of each animal through the arthritis visual score from D7 (as illustrated in FIG. 16). The reaction was different between animals and did not systematically concern the 4 limbs. The arthritis score increased from D7 to D24 to decrease on D28. The variations observed between groups and over time were statistically significant, The Coll2-1 peptide also induced an increase in the arthritis score. On D17, the difference between Groups 3 and 4 and Group 7 was statistically significant (p=0.0332 and p=0.0072 respectively). On D21, Group 1 was statistically different from Group 4 and Group 7 (p=0.0041 and p=0.0450 respectively). On D24, the difference between Group 1 and Group 4 was also statistically significant (p=0.0080).
II. Knee edema (diameter)

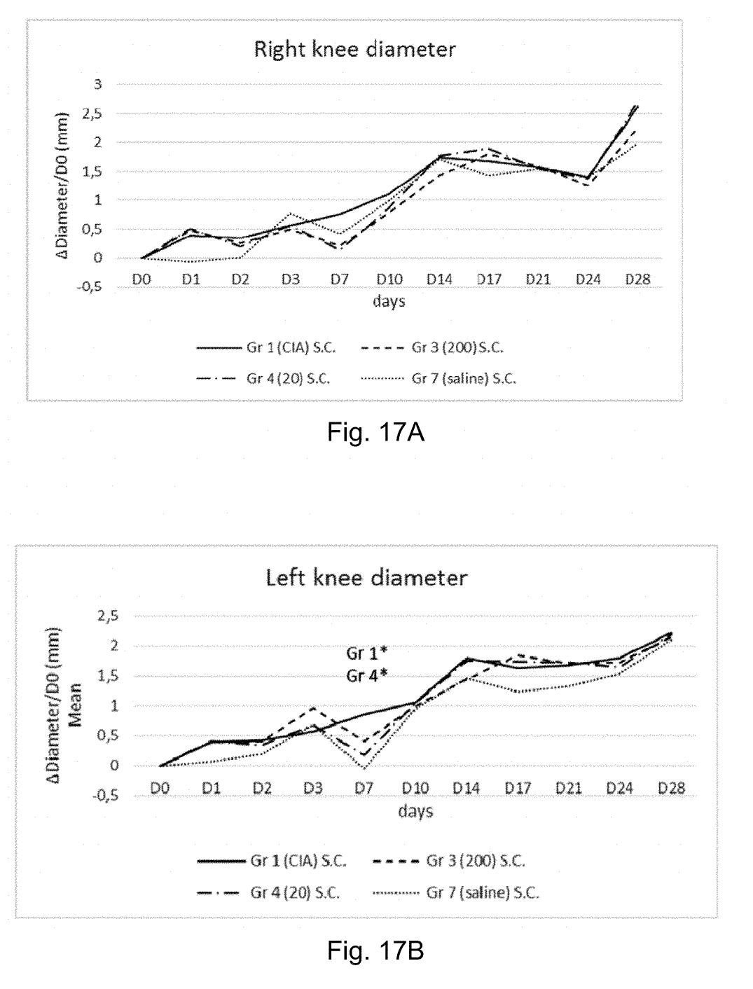

Knee edema (diameter) measured with the digital caliper (in mm) was analyzed considering the variation of knee diameter compared to D0 (Δdiameter/D0). The variation was expressed in mm. Right knee diameter increased from D7 in all groups (as illustrated in FIG. 17A). Left knee also increased but in a lesser extent (FIG. 17B). It was not possible to discriminate the groups based on this evaluation. However the variations over time for the right knee and over time and between groups were significant.
III. Histological analysis

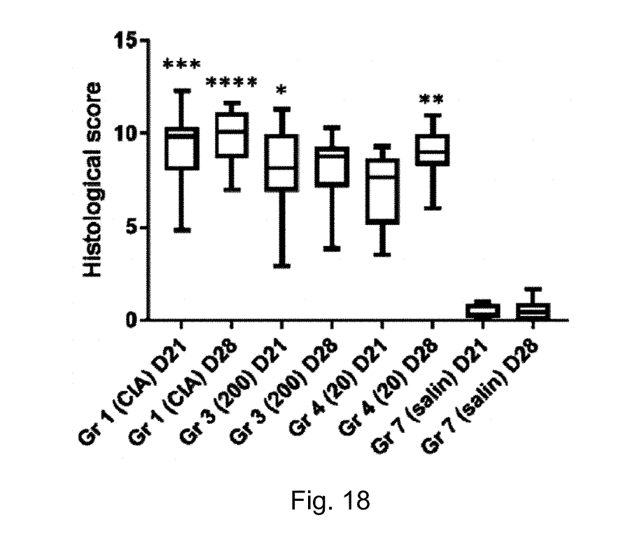

Right and left knees were considered together for the histological analysis after S.C. injection. Type II bovine collagen (Group 1, CIA) induced a significant increase in the global histological score compared to saline (Group 7) (p=0.0005 on D21 and p<0.0001 on D28) (as illustrated in FIG. 18). The injection of Coll2-1 peptide also induced a significant increase in the score on D21 with Group 3 (200 μg) (p=0.0252 versus Gr7) and on D28 with Group 4 (20 μg) (p=0.0025 versus Group 7).

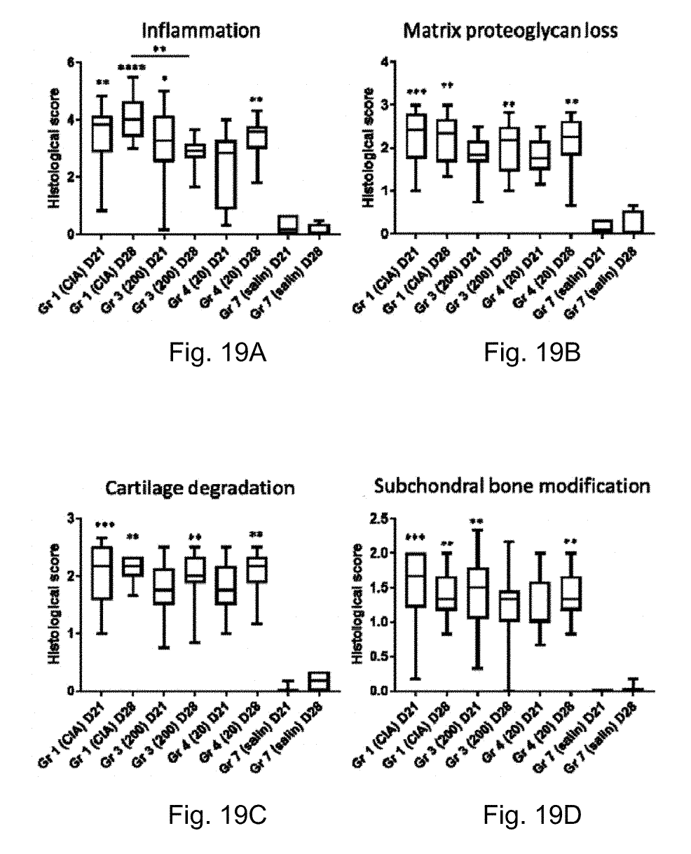

When considering the evaluated criteria separately, type II bovine collagen (Group 1, CIA) induced a significant increase of them on both D21 and D28 (FIG. 19). Group 1 induced a significant increase of the inflammatory criteria (p=0.0017 on D21 and p=0.0008 on D28 versus Gr7). Matrix proteoglycan loss was also significantly increased in Gr1 (p=0.0004 on D21 and p=0.0011 on D28 versus Gr7). Type II bovine collagen (Gr1) induced a significant modification of articular cartilage when compared to saline (Gr7) on both D21 and D28 (p=0.0005 and p=0.0016 respectively) and a significant modification of the subchondral bone as well (p=(3.0004 on D21 and p=0.0082 on D28).

The injection of Coll2-1 peptide at both doses (200 and 20 μg) also induced significant changes in the evaluated criteria, These significant changes were more pronounced on D28:
Significant increase in inflammatory criteria with 200 μg on D21 (p=0.0217) and 20 μg (Gr4) (p=0.0021 on 028);
Significant increase of the loss of matrix proteoglycan with both doses on D28 (p=0.0072 Gr3 and p=0.0024 Gr4);
Significant increase in the cartilage degradation criteria with both doses on D28 (p=0.0070 Gr3 and p=0.0024 Gr4) and
Significant increase in the subchondral bone modification score with Gr3 on D21 (p=0.0025) and with Gr4 on D28 (p=0.0065).

In conclusion, in the collagen arthritis model, both the injection of type II collagen bovine and the injection of the Coll2-1 peptide cause an increase in the histological score. When we consider the parameters evaluated separately, we find that the peptide leads to modifications similar to those induced by the injection of collagen, namely, reduction of the content of the matrix in proteoglycans and lesions of the articular cartilage and the subchondral bone

### Results According to SCW (Streptococcal Cell Walls) Model—I.A Injection

I. Arthritis score

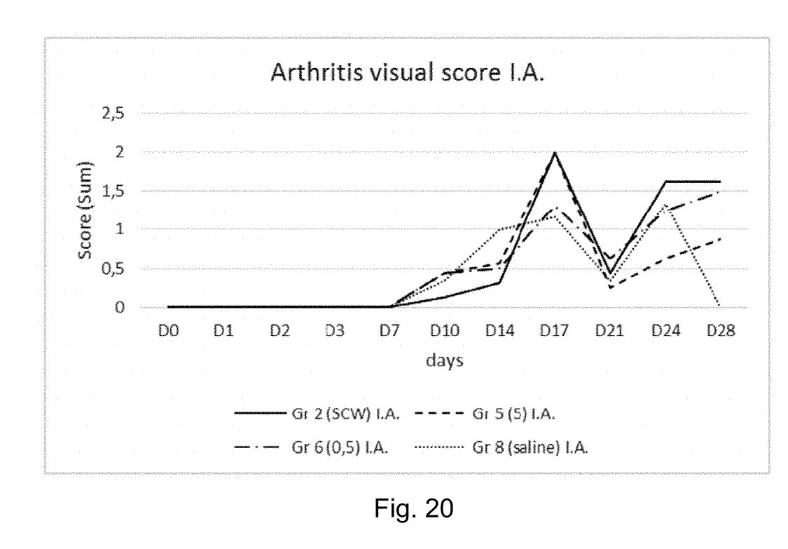

The injection of SCW induced a reaction that was evaluated through the arthritis visual score from D10 (FIG. 20). The arthritis score increased from D10 to reach a peak on D17. The Coll2-1 peptide also induced an increase in the arthritis score. Gr2 (SOW) and Gr5 (Coll2-1 peptide 5 μg) exhibited a very similar evolution for the arthritis score. The variations observed over time and the interaction between parameters were statistically significant. On D17, Gr 8 (saline) was statistically different from Groups 2 (SCW) and 5 (Coll2-1 peptide 5 μg) (p=0.0421 and p=0.0421 respectively). The later were also statistically different from Gr 6 (Coll2-1 peptide 0.5 μg) (p=0.0174 and p=0.0174 respectively). On D24, Gr2 and Gr5 were statistically different (p=0.0134), On D28, Gr2 (SCW) and Gr6 (Coll2-1 peptide 0.5 μg) were statistically different from Gr8 (saline) (p=0.0017 and p=0.0046 respectively).
II. Knee edema (diameter)

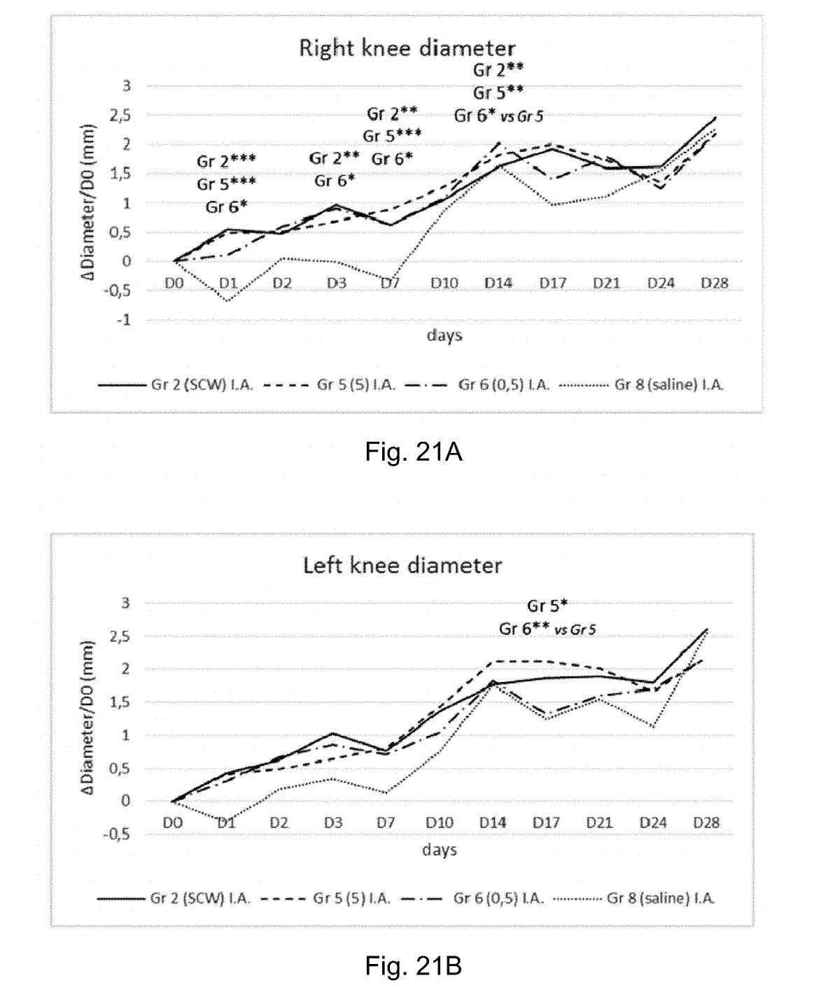

Knee edema (diameter) measured with the digital caliper (in mm) was analyzed considering the variation of knee diameter compared to D0 (Δdiameter/D0). The variation was expressed in mm. Both knee diameter increased from D0 in all groups (FIG. 21). It was possible to discriminate the groups based on this evaluation. The variations over time and between groups were significant.

The right knee diameter allowed to discriminate the study groups. Gr2 (SCW), Gr5 (Coll2-1 peptide 5 μg) and Gr6 (Coll2-1 peptide 0.5 μg) were significantly different from Gr8 (saline) on D1 (p=0.0004, p=0.0007 and p=0.0452 respectively). Gr2 and Gr6 remained statistically different from G8 on D3 (p=0.0067 and p=0.0135 respectively). The 3 groups were significantly different from Gr8 on D7 (p=0.0099 Gr2, p=0.0005 Gr5 and p=0.0105 G6). The statistically significance was found again on D17 for Gr2 and Gr 5 versus Gr8 (p=0.0079 and p=0.0035 respectively). At this time point, Gr5 and Gr6 were statistically different (p=0.0326).

Even if the diameter of the left knee also increased, it was not possible to discriminate the group as with the right knee evolution. The only statistical significance was recorded on D17 where Gr 5 was statistically different from Gr2 and Gr8 (p=0.0050 and p=0.0333 respectively).
III. Histological Analysis

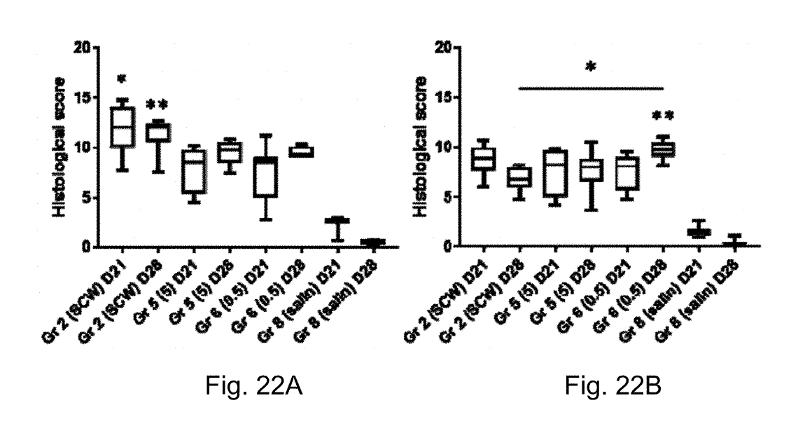

Data were considered for right and left knees separately in the case of I.A. injection. The study groups presented different global histological score (FIG. 22). The strongest difference between groups was observed on the right knee (injected). Gr2 (SCW) exhibited a significantly higher global histological score than Gr8 (saline) on both D21 (p=0.0119) and D28 (p=0.0045). However not significant Coll2-1 peptide (Gr5 and Gr6) exhibited a global histological score intermediate between Gr8 (saline) and Gr2 (SCW). On the left knee the differences were not significant except for Gr6 (Coll2-1 peptide 0.5 μg on D28 (p=0.0022 versus Gr8 and p=0.0499 versus Gr2).

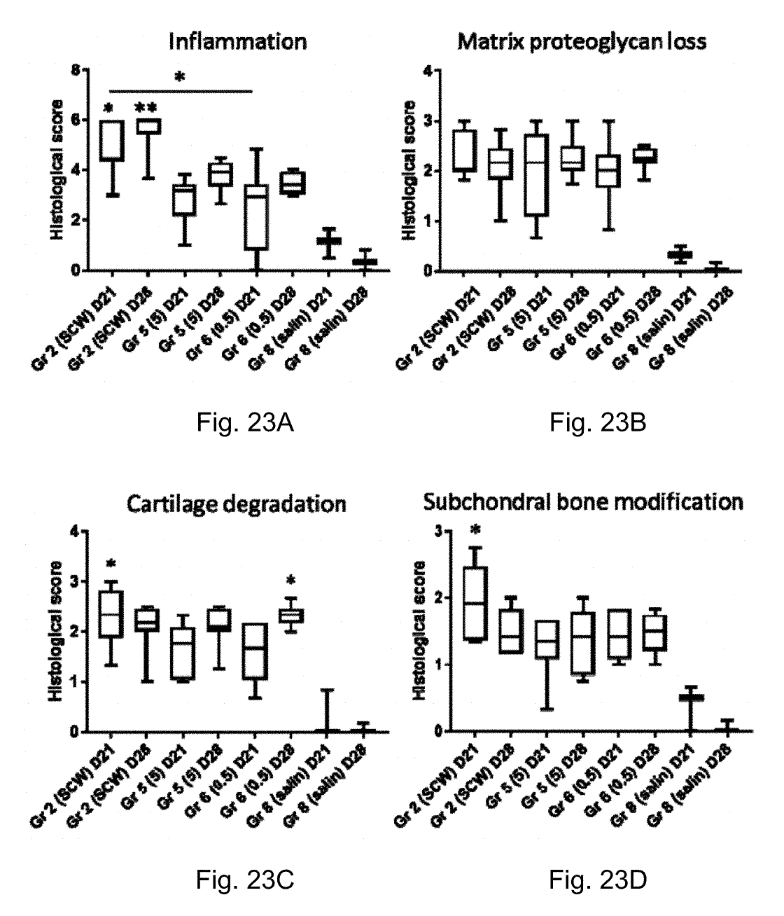

When considering the criteria of evaluation separately in the right knee (FIG. 23), Gr2 (SCW) showed a significant difference in the inflammatory parameter on both D21 and D28 (p=0.0263 and p=0.0020 respectively versus Gr8). Coll2-1 peptide induced an intermediate increase in the inflammatory criteria on D28 however not statistically significant. The effect of SCW (Gr2) was also seen on cartilage degradation and subchondral bone modification. Interestingly Gr6 (Coll2-1 peptide 0.5 μg) induced a significant increase in cartilage degradation score on D28 (p=0.0201 versus Gr8).

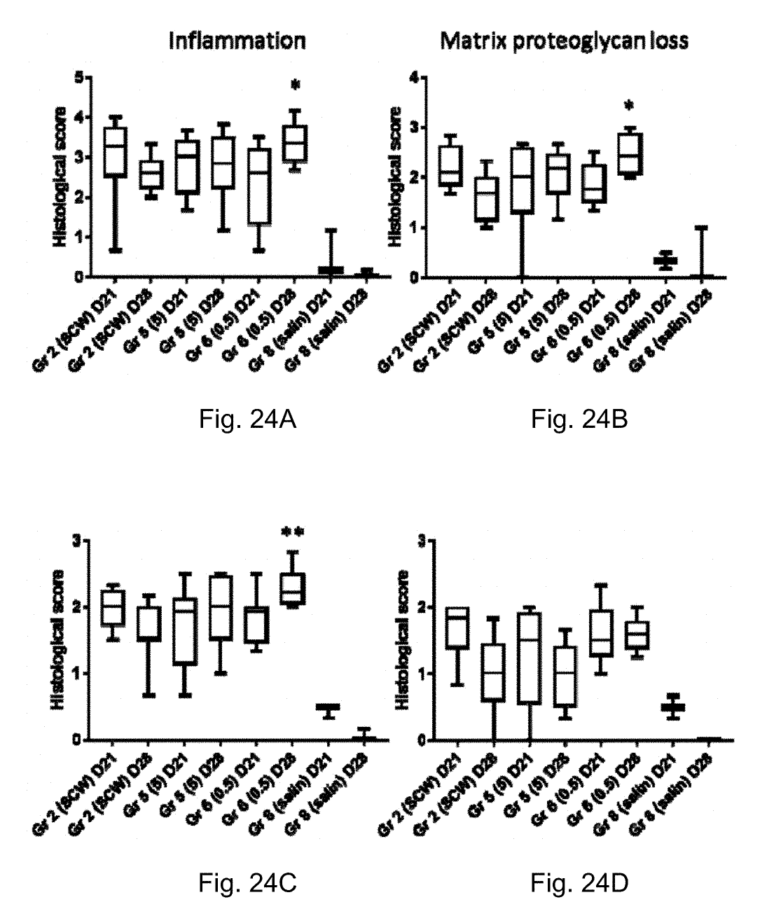

When considering separately the criteria of evaluation on the left knee (FIG. 24) results were more variable. Only Gr6 produced a significant increase in inflammatory (p=0.0118 versus Gr8), matrix proteoglycan loss (p=0.0131 versus Gr8) and cartilage degradation (p=0.0024) scores.

In conclusion, In the SCW arthritis model, the different groups show histological scores of different degrees. The score obtained between D21 and D28 show an evolution between the two evaluation times. The SCW group had the highest score and the “immunogenic peptide” groups had intermediate scores, located between the SCW group and the salt control group. In the same way, when we consider independently the parameters composing the global histological score, namely inflammation, proteoglycan depletion, loss of articular cartilage and bone erosion, the SCW group shows the most important lesions. However, the “immunogenic peptide” groups again have an intermediate score. Thus, both SCW or peptide injections decrease the proteoglycan content of the cartilage and induce lesions of the articular cartilage and subchondral bone

1. A medicament comprising as active ingredient an inhibitor of Coll2-1 peptide activity and/or an inhibitor of Coll2-1NO₂ peptide activity for use in the prevention and/or treatment of osteoarthritis.

2. A medicament comprising as active ingredient an inhibitor of Coll2-1 peptide activity and/or an inhibitor of Coll2-1NO₂ peptide activity for use in the prevention and/or treatment of rheumatic and musculoskeletal diseases (RMDs).

3. The medicament according to claim 1, wherein the Coll2-1 peptide activity and/or an inhibitor of Coll2-1NO₂ peptide activity is due to Coll2-1 peptide and Coll2-1NO₂ peptide, respectively, or a peptide comprising Coll2-1 peptide and Coll2-1NO₂ peptide, respectively.

4. The medicament according to claim 1, wherein the inhibitor of Coll2-1 or Coll2-1NO₂ peptide activity inhibits or reduces the enhancing effects of Coll2-1 or Coll2-1NO₂ peptide activity on any one or more or all of the following: the H₂O₂ production, the expression of angiogenic factors, the expression of IL-6 and 1L-8 each in osteoarthritic synovial fibroblast cells.

5. The medicament according to claim 1, wherein the inhibitor of Coll2-1 or Coll2-1NO₂ peptide activity is an immunological binding partner which is immune-reactive with an epitope comprised in the amino acid sequence HRGYPGLDG (SEQ ID NO: 1).

6. The medicament according to claim 5, wherein the immunological binding partner is selected from the group consisting of polyclonal antibodies, monoclonal antibodies, humanized antibodies, Fc fragments, Fab fragments, single chain antibodies (scFv), chimeric antibodies, biobetters or other antigen-specific antibody fragments specifically binding to Coll2-1 or Coll2-1NO₂ peptide.

7. The medicament according to claim 5, wherein the immunological binding partner is monoclonal or polyclonal antibody specifically binding to Coll2-1 or Coll2-1NO₂ peptide.

8. The medicament according to claim 5, wherein the medicament comprises a mixture of an immunological binding partner specifically binding to Coll2-1 peptide and an immunological binding partner specifically binding to Coll2-1NO₂ peptide.

9. A method of prevention and/or treatment of osteoarthritis comprising the administration of a subject in need thereof an effective dose of an inhibitor of Coll2-1 peptide activity and/or an inhibitor of Coll2-1NO₂ peptide activity.

10. A method of prevention and/or treatment of rheumatic and musculoskeletal diseases (RMDs) comprising the administration of a subject in need thereof an effective dose of an inhibitor of Coll2-1 peptide activity and/or an inhibitor of Coll2-1NO₂ peptide activity.

11. The method according to claim 9, wherein the active ingredient is an immunological binding partner which is immune-reactive with an epitope comprised in the amino acid sequence HRGYPGLDG (SEQ ID NO: 1).

12. The method according to claim 10, wherein the active ingredient is an immunological binding partner which is immune-reactive with an epitope comprised in the amino acid sequence HRGYPGLDG (SEQ ID NO: 1).
